(19) United States
(12) Patent Application Publication
(10) Pub. No.: US 2022/0336098 A1
(43) Pub. Date: Oct. 20, 2022

(54) SETTING A DATA EVENT PARAMETER FOR AN IMPLANTABLE MEDICAL DEVICE BASED ON A CARE USAGE PATTERN

(71) Applicant: INSPIRE MEDICAL SYSTEMS, INC., Golden Valley, MN (US)

(72) Inventors: John Rondoni, Plymouth, MN (US); David Dieken, Minneapolis, MN (US)

(73) Assignee: INSPIRE MEDICAL SYSTEMS, INC., Golden Valley, MN (US)

(21) Appl. No.: 17/723,821

(22) Filed: Apr. 19, 2022

Related U.S. Application Data
(60) Provisional application No. 63/176,518, filed on Apr. 19, 2021.

(51) Int. Cl. G16H 40/67; H04W 4/38; H04W 74/06; H04W 72/08

(52) U.S. Cl. CPC G16H 40/67; H04W 4/38; H04W 74/06; H04W 72/087

(57) ABSTRACT

A system and/or method involving identifying a care usage pattern of an implantable medical device (IMD) for a patient associated with the IMD, and setting a data event parameter for the IMD based on the care usage pattern.

### CROSS-REFERENCE TO RELATED APPLICATIONS

This application claims the benefit of the filing date of U.S. Provisional Application Ser. No. 63/176,518, filed Apr. 19, 2021 and entitled “Setting a Data Event Parameter for an Implantable Medical Device Based on a Care Usage Pattern,” the entire teachings of which are incorporated herein by reference.

### BACKGROUND

Many implantable medical devices process data and/or communicate with external circuitry, such as consumer devices. The external circuitry may be used to provide data to the patient or to a medical caregiver, such as for reporting diagnostics, activating care, adjusting care, and/or other purposes. Processing data and/or communicating data with external circuitry may result in battery depletion and/or other problems with the implanted medical device.

### BRIEF DESCRIPTION OF THE DRAWINGS

FIG. 1 is a flow diagram schematically representing an example method comprising setting a data event parameter based on a care usage pattern.

FIG. 2 is a block diagram schematically illustrating an example implantable medical device (IMD) system.

FIGS. 3A-3B are diagrams schematically representing deployment of an example IMD.

FIGS. 4A-4B are block diagrams schematically illustrating example IMDs, which include an implantable sensor arrangement.

FIGS. 5A-8D are diagrams, which may comprise part of and/or are example implementations of example methods.

FIGS. 9A-9C are block diagrams schematically illustrating an example care usage engine of an IMD system.

FIGS. 10A-13B are diagrams, which may comprise part of and/or are example implementations of example methods.

FIGS. 14A-14D are graphs illustrating example configurable polling intervals.

FIGS. 15A-15C are diagrams, which may comprise part of and/or are example implementations of example methods.

FIG. 16 is a diagram illustrating an example method for setting a configurable polling interval, which may comprise part of and/or is an example implementation of example methods.

FIGS. 17A-20C are diagrams, which may comprise part of and/or are example implementations in example methods.

FIGS. 21A-21C are graphs illustrating example configurable time windows for data processing.

FIG. 22 is a diagram illustrating an example method for setting a configurable time window for performing data processing, which may comprise part of and/or is an example implementation of example methods.

FIG. 23 is a diagram, which may comprise part of and/or is an example implementation of example methods.

FIG. 24 is a block diagram schematically representing example data model types.

FIG. 25 is a block diagram schematically representing at least some example known input sources.

FIG. 26 is a diagram schematically representing an example method of constructing a data model for use in later setting a configurable data event parameter of an IMD.

FIG. 27 is a diagram schematically representing an example method of using a constructed data model for setting the configurable data event parameter using internal measurements.

FIG. 28 is diagram schematically representing an example method of constructing a data model.

FIG. 29 is a diagram schematically representing an example method of using a constructed data model for setting a data event parameter.

FIGS. 30A-30B are block diagrams schematically representing example IMD systems including a care usage engine.

FIG. 31 is a diagram including a front view of an example device (and/or example method) implanted within a patient's body.

FIG. 32 is a diagram schematically representing an example IMD.

FIG. 33A is a block diagram schematically representing an example control portion.

FIG. 33B is a diagram schematically illustrating at least some example arrangements of a control portion.

FIG. 34 is a block diagram schematically representing a user interface.

FIG. 35 is a block diagram which schematically represents some example implementations by which an IMD may communicate wirelessly with external circuitry outside the patient.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to methods involving identifying a care usage pattern of an implantable medical device (IMD) for a patient associated with the IMD and setting a data event parameter for the IMD based on the care usage pattern.

At least some examples of the present disclosure are directed to devices, systems, and/or methods for controlling a data event of an IMD system, including an IMD implanted within a patient, by setting a data event parameter based on an identified care usage pattern of the IMD. The care usage pattern may comprise at least one cycle of expected care, predicted care (e.g., based on past observation of the patient) and/or currently occurring care provided to the patient by the IMD, herein generally referred to as “care cycles”. In some examples, the care usage pattern may comprise a plurality of different care cycles which are expected or predicted to occur based on different sets of factors. Example factors include an activity pattern of the patient or representative patients, a day of week (e.g., weekday verses a weekend), a time of year, a time of day, among other example factors. In some examples, the IMD may update the care usage pattern, and the associated care cycles, over time based on sensed data.

In at least some examples, the data event parameter comprises a polling interval for data communications and/or a time window for performing data processing operations by the IMD. By setting, and optionally adjusting, the data event parameter based on the care usage pattern, the IMD may optimize energy consumption performance, battery delivery capacity, and/or communication latency. In at least some examples, a polling interval, as used herein, comprises and/or refers to a period of time between successive instances of activation of telemetry circuitry of the IMD to detect a communication signal received from external circuitry. In at least some examples, the time window for performing data processing operations comprises and/or refers to a time of day the IMD initiates and/or performs the data processing operation and/or a length of time the data processing operation may occur.

In some examples, the IMD may adjust a polling interval with respect to different times of the day to balance energy consumption (or power performance) and/or battery delivery capacity with communication latency. In some examples, the care usage pattern may be used to predict when the IMD is performing care and when the patient may communicate with the IMD via external circuitry, and based on the predictions, set configurable and adjustable polling intervals to balance energy consumption and/or power performance with communication latency.

In some examples, the IMD may set and/or adjust at least one time window for performing time-insensitive data processing operations to optimize battery delivery capacity. For example, the care usage pattern may be used to predict when the IMD may exhibit a battery parameter that is below or above a threshold level, such as a battery power delivery capacity of 2.5 volts (V). The battery parameter may be impacted by the IMD providing care, as further described herein. As such, the prediction of the value of the battery parameter may comprise a prediction of care being provided by the IMD. Based on the prediction(s), the IMD may set and/or adjust at least one time window to occur for performing time-insensitive data processing operations, such as setting the time window during a time of the day that the value of the battery parameter is predicted to be within (e.g., above or below) the threshold level, such as a power threshold.

In some examples, the devices, systems, and methods of the present disclosure are configured and used for sleep disordered breathing (SDB) care, such as obstructive sleep apnea (OSA) care, which may comprise monitoring, diagnosis, and/or stimulation therapy. However, in other examples, the system is used for other types of care and/or therapy, including, but not limited to, other types of neurostimulation or cardiac care or therapy. In some examples, such other implementations include therapies, such as but not limited to, central sleep apnea, multiple-type sleep apnea, cardiac disorders, pain management, seizures, deep brain stimulation, respiratory disorders, and various combinations thereof.

It will be further understood that in some instances, a data model may be used to identify some of the internally sensed inputs and/or some of the ways in which the internally sensed inputs may be used to set the data event parameter. Non-data model techniques may be used with (or without) the data model techniques to determine the desired internally sensed inputs.

Accordingly, it will be further understood that aspects of the various example methods involving non-data models and those involving data models may be selectively mixed and matched with each other as desired to achieve the desired and/or effective manner of identifying the care usage pattern of the IMD and/or setting a data event parameter based on the care usage pattern.

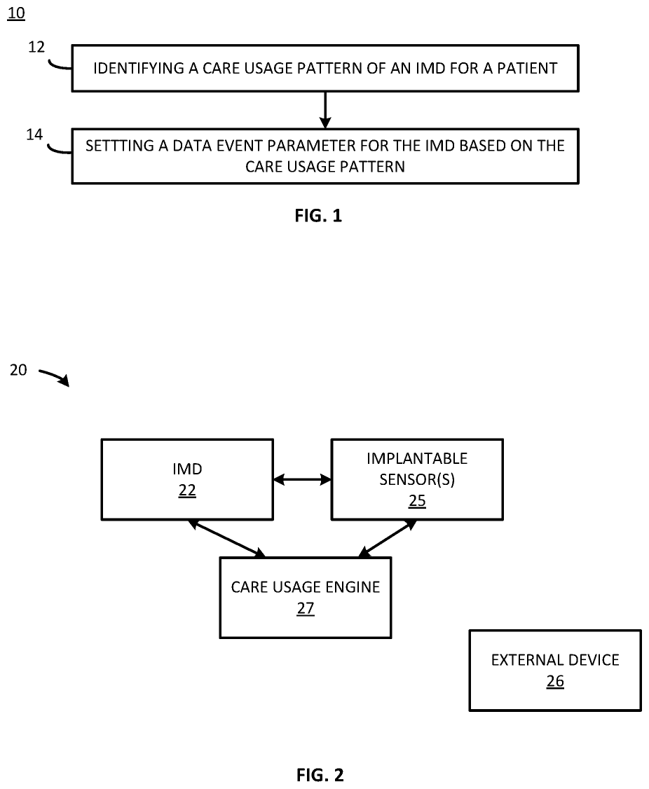

These examples, and additional examples, are described in association with at least FIGS. 1-35.

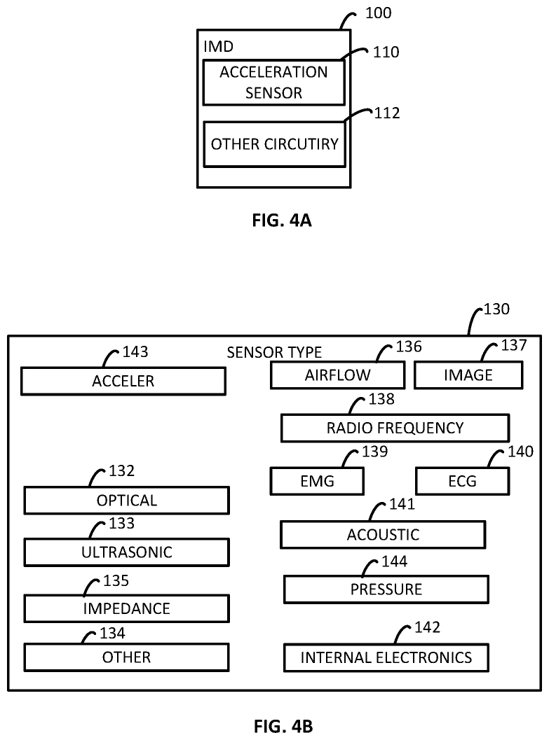

FIG. 1 is a flow diagram schematically representing an example method 10 comprising setting a data event parameter based on a care usage pattern. The method 10 comprises identifying a care usage pattern of an IMD for a patient associated with the IMD, as shown at 12 in FIG. 1, and setting a data event parameter for the IMD based on the care usage pattern, as shown at 14. As further illustrated by FIG. 2, the IMD may form part of an IMD system that includes the IMD and at least one implantable sensor, which may form part of the IMD or is otherwise in communication with the IMD. The at least one implantable sensor may comprise an acceleration sensor, an impedance sensor, an acoustic sensor, an optical sensor, a pressure sensor, among other types of sensors and various combinations thereof, examples of which are further illustrated by at least FIGS. 4A-4B.

The data event parameter may be associated with a data event performed by the IMD, such as a data communication and/or data processing. In some examples, setting the data event parameter may comprise setting a polling interval for data communication by the IMD and/or setting a time window for performing data processing by the IMD. In at least some examples, as used herein, a data event comprises and/or refers to an activity implemented by or using an IMD, such as a listening event, data communication, and/or data processing. In some examples, a data event may occur over a period of time. In some examples, a data event may be a singular event. In some examples, a data event may be a series of multiple events.

Various examples herein refer to different types of “time”, such as time of day, length of time, and time window. In some examples, as used herein, a time of day comprises and/or refers to a time as indicated by a clock (e.g., 4 pm), such as a time to start or stop the data event or data event parameter. In some examples, as used herein, a length of time comprises and/or refers to a duration, such as a duration for setting the polling interval for performing data processing operations (e.g., one hour, two hours, twenty minutes). In some instances, a length of time is herein interchangeably referred to as a period of time or a duration. In some examples, a time window may comprise and/or refer to both the time of day and the length of time (e.g., from 7 am to 8 am).

In some examples, the IMD may communicate data with various external circuitry for monitoring care and patient control. In some instances, the communication may be accomplished using specialized communication schemes, such as Medical Implant Communication Service (MICS). In some examples, the IMD may communicate with a consumer device, such as a smartphone, using standard communication protocols. Example communication protocols include Bluetooth, Bluetooth Low Energy (BLE), ZigBee, Z-wave, Long-Term Evolution (LTE), among other types of standard communication protocols. Robust and low latency communication over several meters may be power intensive due to the energy for the data communication as well as the energy for the IMD to listen for an external device to initiate a communication session. In some examples, a listening event may comprise an event during which the IMD powers the telemetry circuitry, listens for a communication, and then acts upon the communication or shuts the telemetry circuitry down. In some examples, the interval between listening events is herein referred to as the polling interval. Adjusting the polling interval with respect to particular times of the day may be used to balance communication latency (e.g., the speed at which the IMD responds to a request for data communication from an external device) and energy consumption and/or power performance.

However, examples are not limited to setting a polling interval based on the care usage pattern. In some examples, the data event parameter setting may be a time window for performing data processing. The data to be processed may be queued for a period of time, such as hours and/or days, and then processed during a period of time during which lower power demand occurs. For example, the IMD may immediately (or within a threshold period of time) process time-sensitive data operations and perform time-insensitive data operations in batches during the time period of lower power demand in order to balance and/or improve energy consumption and/or power management and/or ensure that battery power capacity is not exceeded during time-sensitive operations of the IMD, as further described herein.

The following provides illustrative and non-limiting examples of setting data event parameters. As an example, at the start of the time window associated with a data event parameter comprising a first polling interval of ten seconds, the IMD may transition to or execute a ten second polling interval (e.g., every ten seconds, the IMD initiates a listening event). At the end of the time window, the IMD may transition to a second polling interval that is greater than the first polling interval (e.g., ten minutes). As another example, at the start of the time window associated with a data event parameter comprising a time window for performing data processing, the IMD may begin processing batched data. At the end of the time window, if the data processing is not complete, the IMD may stop processing until another data processing time window is reached.

In some examples, the data event parameter may be adjusted based on the care usage pattern. In at least some examples, the care usage pattern, as used herein, is a pattern indicative of care expected to, predicted to (e.g., based on observed data), and/or currently being provided by the IMD to the patient, which may be associated with different times of the day. The method 10 may further comprise configuring the IMD to include the care usage pattern, such as storing the care usage pattern on memory of the IMD.

As described above, the care usage pattern may comprise at least one care cycle. In some examples, as further described herein, the care usage pattern may comprise expected care cycles of the IMD, observed care cycles of the IMD and/or a current care cycle of the IMD. In some examples, as used herein, expected care cycles of the IMD comprise and/or refer to predicted care usage time(s) and/or amount of care provided to the patient by the IMD based on data from external data sources. The expected care cycles may be based on literature data, input from a medical caregiver, demographic data associated with the patient and/or a representative plurality of patients, and input from the patient, among other data which may be used to predict when care is to be provided to the patient. In some examples, observed care cycles of the IMD comprise and/or refer to predicted care usage time(s) and/or amount of care provided to the patent by the IMD based on internally obtained data and/or observed care cycles of the IMD. The observed care cycle may be based on data sensed by the IMD and/or otherwise internal to the IMD, such as physiological data and/or care usage data. In some examples, a current care cycle of the IMD comprises and/or refers to presently or a real-time care event of the IMD, which may be based on physiological data sensed by the IMD and/or an implantable sensor in communication with the IMD.

In some examples, the IMD may identify which care cycle to use based on a set of factors, which may be internally sensed and/or input from external sources. Example factors include activity pattern of the patient or representative patients, day of the week, time of year, time of day, among other factors. For example, the activity pattern of the patient may comprise or be indicative of an amount or type of movement (e.g., did the patient exercise or not, at work all day, etc.) which may impact care provided by the IMD. Other example factors may include a sleep pattern, dietary intake, pharmaceutical medications, and weather, among others. However, examples are not so limited, and in some examples, the care usage pattern may comprise a single care cycle. In some examples, the IMD may determine or identify at least a portion of the set of factors based on data sensed by the IMD, as further described herein.

In some examples, the care usage pattern may comprise different combinations of the expected care cycles of the IMD, the observed care cycles of the IMD, and the current care cycle of the IMD. For example, the IMD may be configured to set the data event parameter based on the expected care cycles initially, and may transition to the observed care cycles over time and/or to the current care cycle in response to real-time data which may override the data event parameter setting based on the expected or observed care cycles. In some examples, a care usage pattern may comprise combinations of expected care cycles and observed care cycles, which are each associated with a different set of factors.

As further described herein, the method 10 may include a number of additional steps and/or variations, such as performing the data event based on the data event parameter setting. Performing the data event based on the data event parameter setting may optimize energy consumption by the IMD and/or optimize battery delivery capacity. In some examples, the data event parameter setting may be used to balance or optimize communication latency and energy consumption.

FIG. 2 is a block diagram schematically illustrating an example IMD system 20. The IMD system 20 includes an IMD 22, at least one implantable sensor 25, a care usage engine 27, and an optional external device 26. Details on the various components are provided below.

In general terms, the IMD 22 is configured for implantation into a patient, and is configured to provide and/or assist in providing care to the patient. The at least one implantable sensor 25 may assume various forms, and is generally configured for implantation into the patient and to sense at least one of physiological data and care usage data, as further described herein. In some examples, the at least one implantable sensor 25 includes a sensor component in the form of or akin to a motion-based transducer. The motion-based transducer sensor component of the at least one implantable sensor 25 may be or includes an acceleration sensor such as an accelerometer (e.g., a multi-axis accelerometer such as a three-axis or six-axis accelerometer), a gyroscope, etc. In some examples, the at least one implantable sensor 25 includes more than one sensor, such as an acceleration sensor and non-acceleration sensor circuitry. The at least one implantable sensor 25 may be carried by the IMD 22, may be connected to the IMD 22, or may be a standalone component not physically connected to the IMD 22, as further described herein.

The care usage engine 27 is programmed to perform one or more operations as described below and based on a care usage pattern. In some examples, the care usage engine 27 identifies a care usage pattern (e.g., selects a care cycle) and sets a data event parameter based on the identified care usage pattern. In some examples, the care usage engine 27 may identify the care usage pattern based on a set of factors, as described above. In some examples, the care usage engine 27 identifies the care usage pattern based on data sensed via the at least one implantable sensor 25 (e.g., based on an output of the at least one implantable sensor 25 which is input to the care usage engine 27). In some examples, the care usage engine 27 receives the care usage pattern (e.g., expected care cycles) and/or data from the at least one implantable sensor 25 and is programmed (or is connected to a separate engine that is programmed) to set the data event parameter based, at least in part, on the input care usage pattern and/or data from the at least one implantable sensor 25.

In some examples, the care usage engine 27 is programmed (or is connected to a separate engine that is programmed) to affect (or not effect) one or more features or the like relating to operation of the IMD system 20 in response to setting the data event parameter. The care usage engine 27 may reside partially or entirely with the IMD 22, partially or entirely with the external device 26, or partially or entirely with a separate device or component (e.g., the cloud, etc.). Where provided, the external device 26 may wirelessly communicate with the IMD 22, and is operable to facilitate performance of one or more operations as described below. For example, the external device 26 may be used to initially program the IMD 22, and the IMD 22 then processes information and delivers care independent of the external device 26.

In some examples, the external device 26 may be omitted. In some such examples, the IMD 22, the at least one implantable sensor 25 and the care usage engine 27 perform one or more of the operations described below without the need for the external device 26 or human input. The care usage engine 27 may be further programmed to provide information to the patient and/or caregiver, such as processed data or other information of possible interest implicated by information from the at least one implantable sensor 25. In some examples, the care usage engine 27 may provide information indicating the data event parameter to another engine of the IMD 22 that is programmed to execute the data event based on the data event parameter.

The care usage engine 27 (or the logic akin to the care usage engine 27) may be incorporated into a distinct engine or engine programmed to perform certain tasks. For example, the logic of the care usage engine 27 as described below may be part of a care engine and utilized in controlling care provided to the patient, such as but not limited to stimulation therapy delivered to the patient. Logic embodied by the care usage engine 27 may identify or detect a care usage pattern of the IMD 22 in various manners. In some examples, the care usage pattern may be recognized by a function that references expected care cycles and/or data sensed by the at least one implantable sensor 25. As an example, if the data from the at least one implantable sensor 25 includes a pattern of a particular expected care cycles, then the expected care cycle is identified. In some examples, the care usage pattern may be recognized with reference to data from the at least one implantable sensor 25, data from external data sources, and/or a data model (e.g., modeling or artificial intelligence or artificial learning). For example, one or more data sources (including data from the at least one implantable sensor 25) may be employed in a probabilistic decision model to recognize or identify cycles of expected and/or observed care cycles and a currently occurring care cycle.

With these and related examples, the care usage engine 27 is programmed to evaluate the probability of care being provided at different times of the day, and deem or decide when care is expected, observed, and/or is currently occurring (and when not). As an example, care expected or observed to be occurring may be recognized in response to a likelihood of occurrence being greater than a threshold, such as 80 percent or greater. Determining a probability may include weighting different factors and summing the weights to determine the probability. The factors may comprise, but are not limited to, historical data sensed by the at least one implantable sensor 25, as well as patterns identified within the sensed data, and/or other inputs, such as a time of day, day of the week, etc. In some examples, the factors may comprise at least some of the set of factors, as previously described in connection with FIG. 1. The factors may be weighted based on a relevancy of the factors (or relevancy of a value of the factor) to predicting or identifying care being provided by the IMD. In some examples, the factors may be weighted based on whether the respective factor indicates care is about to or is being provided by the IMD or not. As particular examples, a time of day or night may be weighted for or against care being provided, while particular body motion and/or posture patterns and/or other physiological data (e.g., low heart rate) may be weighted to indicate for or against care being provided by the IMD 22. As further described herein, the probability may be revised over time based on additionally obtained data. Similarly, the care usage pattern may be recognized in response to a likelihood of care being provided being greater than a threshold, such as 80 percent or greater. However examples are not so limited and other thresholds may be used, such as 70 percent, 75 percent, 85 percent, or 90 percent.

While the previously described arrangements comprise a probability associated with care being provided by the IMD, examples are not so limited. In some examples, the probability may additionally or alternatively be associated with predicting when data communications may occur and/or associated with a battery parameter of the IMD.

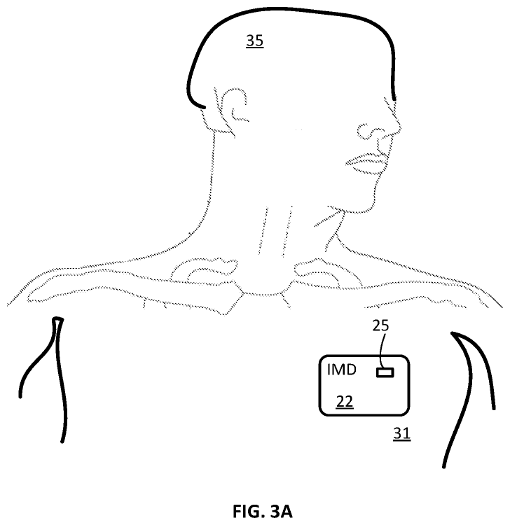

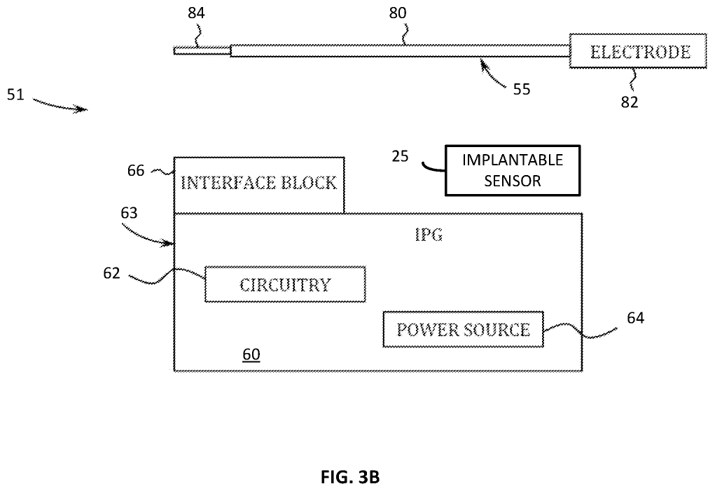

FIGS. 3A-3B are diagrams schematically representing deployment of an example IMD. More specifically, FIG. 3A is diagram including a front view schematically representing deployment of an example IMD 22, which includes at least one implantable sensor 25. As shown in FIG. 3A, in some examples the IMD 22 (and therefore the at least one implantable sensor 25) may be chronically implanted in a pectoral region 31 of a patient 35. However, examples are not so limited and the IMD 22 and/or at least one implantable sensor 25 may be implanted in other regions of a patent, such as the head and/or neck of the patient. In some examples, the at least one implantable sensor 25 comprises an acceleration sensor that senses data including various physiologic phenomenon sensed from this implanted position (e.g., body motion, posture, vibrations, such as anatomy vibrations and device vibrations). The example IMD 22 may be used to implement the method 10 of FIG. 1 in some examples.

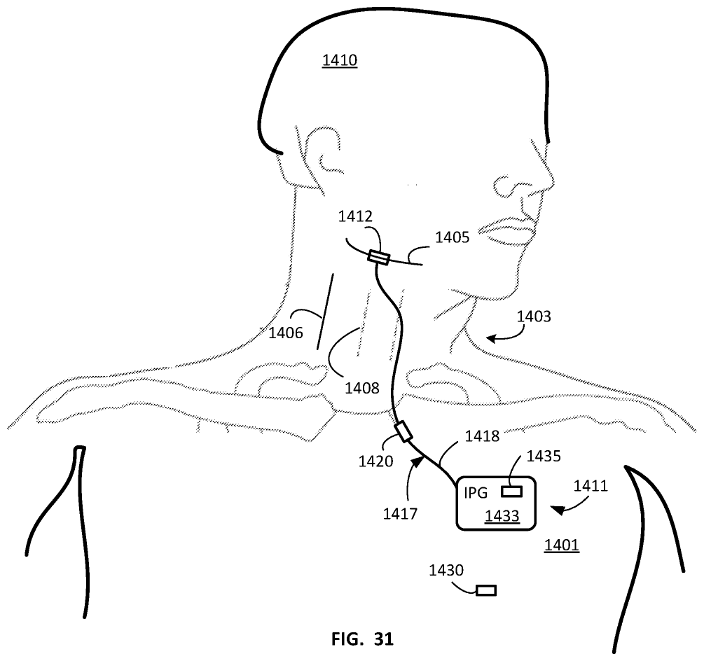

In some examples, the IMD 22 may comprise an implantable pulse generator (IPG), such as for managing sensing and/or stimulation therapy, as later described in association with at least FIGS. 31-35.

FIG. 3B is a block diagram schematically representing one example of an IMD 51 which is an example implementation of, and/or may comprise at least some of substantially the same features and attributes of IMD 22 of the IMD system 20 of FIG. 2. In some examples, the IMD 51 may include an IPG assembly 63 and at least one stimulation lead 55. The IPG assembly 63 may include a housing 60 containing circuitry 62 and a power source 64 (e.g., battery), and an interface block or header-connector 66 carried or formed by the housing 60. The housing 60 is configured to render the IPG assembly 63 appropriate for implantation into a human body, and may incorporate biocompatible materials and hermetic seal(s). The circuitry 62 may include circuitry components and wiring appropriate for generating desired stimulation signals (e.g., converting energy provided by the power source 64 into a desired stimulation signal), for example in the form of a stimulation engine. In some examples, the circuitry 62 may include telemetry components for communication with external circuitry. For example, the circuitry 62 may include a transmitter that transforms electrical power into a signal associated with transmitted data packets, a receiver that transforms a signal into electrical power, a combination transmitter/receiver (or transceiver), an antenna (e.g., an inductive telemetry antenna), etc.

In some examples, the power source 64 includes a battery, such as a rechargeable battery or a primary battery. A rechargeable battery, sometimes referred to as a rechargeable cell or secondary cell, includes and/or refers to an electrical battery which can be charged, discharged to a load (e.g., the IMD 51), and recharged, which may be repeated a number of times. A primary battery, sometimes referred to as a primary cell, includes and/or refers to an electrical battery that is discharged to a load and then may be discarded. A primary battery may not be reused once discharged.

In some examples, the stimulation lead 55 includes a lead body 80 with a distally located stimulation electrode 82. At an opposite end of the lead body 80, the stimulation lead 55 includes a proximally located plug-in connector 84 which is configured to be removably connectable to the interface block 66. For example, the interface block 66 may include or provide a stimulation port sized and shaped to receive the plug-in connector 84.

In general terms, the stimulation electrode 82 may optionally be a cuff electrode, and may include some non-conductive structures biased to (or otherwise configurable to) releasably secure the stimulation electrode 82 about a target nerve. Other formats are also acceptable. Moreover, the stimulation electrode 82 may include an array of contact electrodes to deliver a stimulation signal to a target nerve. In some non-limiting examples, the stimulation electrode 82 may comprise at least some of substantially the same features and attributes as described within at least: U.S. Pat. No. 8,340,785, issued Dec. 25, 2012, and entitled “SELF EXPANDING ELECTRODE CUFF”; U.S. Pat. No. 9,227,053, issued Jan. 5, 2016, and entitled “SELF EXPANDING ELECTRODE CUFF”; U.S. Pat. No. 8,934,992, issued Jan. 13, 2015, and entitled “NERVE CUFF”; and/or U.S. Patent Publication No. 2020/0230412, published on Jul. 23, 2020, and entitled “CUFF ELECTRODE”, the entire teachings of each of which are incorporated herein by reference in their entireties.

In some examples, the lead body 80 is a generally flexible elongate member having sufficient resilience to enable advancing and maneuvering the lead body 80 subcutaneously to place the stimulation electrode 82 at a desired location adjacent a nerve, such as an airway-patency-related nerve (e.g., hypoglossal nerve, phrenic nerve, ansa cervicalis nerve, etc.). In some examples, such as in the case of obstructive sleep apnea, the nerves may include (but are not limited to) the nerve and associated muscles responsible for causing movement of the tongue and related musculature to maintain or restore airway patency. In some examples, the nerves may include (but are not limited to) the hypoglossal nerve and the muscles may include (but are not limited to) the genioglossus muscle. In some examples, lead body 80 may have a length sufficient to extend from the IPG assembly 63 implanted in one body location (e.g., pectoral) and to the target stimulation location (e.g., head, neck). Upon generation via the circuitry 62, a stimulation signal is selectively transmitted to the interface block 66 for delivery via the stimulation lead 55 to such nerves.

The at least one implantable sensor 25 may be connected to the IMD 51 in various fashions. For example, the at least one implantable sensor 25 may include a lead body carrying the motion-based transducer sensor element of an acceleration sensor at a distal end, and a plug-in connector at proximal end. The plug-in connector may be connected to the interface block 66, such as the interface block 66 including or providing a sense port sized and shaped to receive the plug-in connector of the at least one implantable sensor 25, and the lead body extended from the IPG assembly 63 to locate the sensor element at a desired anatomical location. Alternatively, the at least one implantable sensor 25 may be physically coupled to the interface block 66, and thus carried by the IPG assembly 63. In some such examples, the at least one implantable sensor 25 may be considered a component of the IMD 51. In some examples, the physical coupling of the at least one implantable sensor 25 relative to the IPG assembly 63 is performed prior to implantation of those components.

In some examples, the at least one implantable sensor 25 (and in particular, at least the motion-based transducer sensor component as described above) may be incorporated into a structure of the interface block 66, into a structure of the housing 60, and/or into a structure of the stimulation lead 55. With these and similar configurations, the sensor component of the at least one implantable sensor 25 is electronically connected to the circuitry 62 within the housing 60 or other enclosure of the IPG assembly 63. More specifically, the at least one implantable sensor 25 may be connected in various orientations as described within U.S. patent application Ser. No. 16/978,275, filed on Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety. Although the above examples describe an IMD 51 having a stimulation lead 55, examples are not so limited and example IMDs may additionally or alternatively include a lead used for sensing, such as a lead used to sense physiological or other data.

In some examples, the at least one implantable sensor 25 may be wirelessly connected to the IMD 51. In some such examples, the interface block 66 need not provide a sense port for the at least one implantable sensor 25 or the sense port may be used for a second sensor. In some examples, the circuitry 62 of the IPG assembly 63 and circuitry of the at least one implantable sensor 25 communicate via a wireless communication pathway according to known wireless protocols, such as Bluetooth, near-field communication (NFC), MICS, 802.11, etc. with each of the circuitry 62 and the at least one implantable sensor 25 including corresponding components for implementing the wireless communication pathway. In some examples, a similar wireless pathway is implemented to communicate with devices external to the patient's body for at least partially controlling the at least one implantable sensor 25 and/or the IPG assembly 63, to communicate with other circuitry (e.g., other sensors or devices) internally within the patient's body, or to communicate with other sensors external to the patient's body.

As further shown in association with at least FIGS. 4A-4B, the at least one implantable sensor 25 may include an acceleration sensor and/or other sensor circuitry deployed relative to a patient's body. In some examples an acceleration sensor may be implanted internally to sense physiological data such as in a head-and-neck region, a thorax/abdomen region, and/or a peripheral/other region. In some examples, more than one implantable sensor may be implanted in a single region and/or in different multiple regions in the patient's body.

FIGS. 4A-4B are block diagrams schematically illustrating example IMDs, which include an implantable sensor arrangement. As shown in association with at least FIG. 4A, an example IMD 100 includes an acceleration sensor 110 and other circuitry 112, such as (but not limited to) telemetry circuitry.

The acceleration sensor 110 may comprise an accelerometer (e.g., a single axis or multi-axis accelerometer), a gyroscope, a pressure sensor, etc. The acceleration sensor 110 may provide information along a single axis, or along multiples axes (e.g., three-axis accelerometer, three-axis gyroscope, six-axis accelerometer, nine-axis accelerometer, etc. In some examples, an acceleration sensor 110 that provides information along multiple axes may provide information along multiple linear, rotational, and/or magnetic axes, such as three-rotational axes (e.g., a three-axis accelerometer). In some examples, a six-axis acceleration sensor may provide information along three linear axes and three rotational axes. In some examples, a nine-axis acceleration sensor may provide information along three linear axes, three rotational axes, and three magnetic axes. Regardless of an exact form, the sensor component of the acceleration sensor 110 is capable of sensing, amongst other things, information indicative of body motion of the patient, a posture of the patient, and other vibrations. As a point of reference, while information generated by the acceleration sensor 110 is signaled to and acted upon by the IMD 100 (such as by a care usage engine 27 of an IMD 22 of FIG. 2), information from the acceleration sensor 110 may be utilized by other engines, such as by a care engine that manages care or diagnostic data provided to the patient by the IMD, as described below. In some non-limiting examples, the acceleration sensor 110 may form part of the IMD 100 or is otherwise coupled to the IMD 100, as previously described.

The following provides some examples of sensing information indicative of body motion, posture, and vibrations by the acceleration sensor 110, however examples are not so limited and the acceleration sensor 110 may sense body motion, posture, and vibration using a variety of techniques. The acceleration sensor 110 may be used to generate data via sensing of forces in multiple directions or axes. In some examples, the acceleration sensor 110 is a three-axis accelerometer that may sense or measure the static and/or dynamic forces of acceleration on three axes. Static forces include the constant force of gravity. By measuring the amount of static acceleration due to gravity, an accelerometer may be used to identify the angle it is tilted at with respect to the earth. By sensing the amount of dynamic acceleration, the accelerometer may find out how fast and in what direction the IMD is moving, which may be indicative of body movement and, in some examples, indicative of an activity pattern of the patient. Single- and multi-axis models of accelerometers detect magnitude and direction of acceleration (or proper acceleration) as a vector quantity. With these and similar types of sensor constructions, an output from the acceleration sensor 110 may include vector quantities in one, two or three axes.

In some examples, some methods of the present disclosure may include at least some of substantially the same features and attributes as determining or designating a posture of the patient based on data from the acceleration sensor 110 described within U.S. patent application Ser. No. 16/978,275, filed Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety.

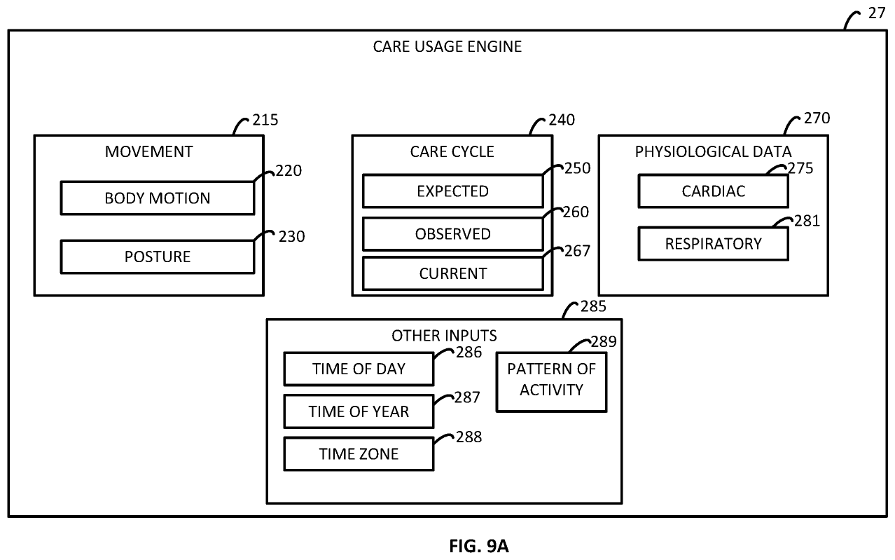

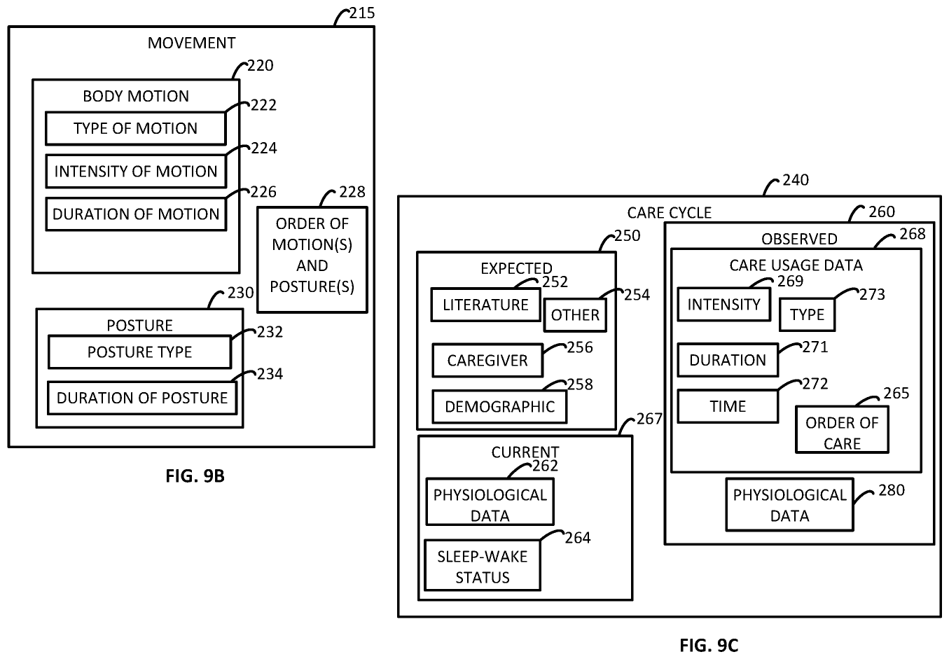

As described above, sensing the amount of dynamic acceleration may be used to identify body motion and posture. Example body motions include movement in a vector or a direction (e.g., walking, running, biking), rotational motions (e.g., twisting), and changes in posture (e.g., change from an upright position to a sitting or supine position), among other movements. The motion may be sensed relative to a gravity vector, such as an earth gravity vector and/or a vertical baseline gravity vector. In some examples, the sensed force(s) may be processed to determine a posture of the patient. As used herein, posture refers to or includes a position or bearing of the body. In some instances, the term “posture” may sometimes be referred to as “body position”. Example postures include upright or standing position, supine position or another generally horizontal body position (e.g., prone, lateral decubitis), a generally supine reclined position, sitting position, etc. Further detail on examples of identifying or determining motion and posture are described below in connection with the example care usage engine 27 of an IMD and sub-engines illustrated in association with at least FIGS. 9A-9C.

In some examples, the acceleration sensor 110 may be used to sense physiological data. The physiological data may include physiological parameters, such as cardiac signals and/or respiration information. In some examples, the respiration information may be determined based on rotational movements of a portion of a chest wall of the patient during breathing. For example, the acceleration sensor 110 may be used to determine respiration information based on rotational movements of a chest wall of the patient as described within U.S. patent application Ser. No. 16/977,664, filed on Sep. 2, 2020, and entitled “RESPIRATION DETECTION”, the entire teachings of which is incorporated herein by reference in its entirety.

The other circuitry 112 may set the data event parameter based on the care usage pattern and perform a data event based on the data event parameter, as described by FIG. 1. For example, the other circuitry may include a processor, memory, and, optionally, telemetry circuitry. In some examples, the processor executes data processing and/or communicates data based on the data event parameter. For example, the processor may activate the telemetry circuitry based on a polling interval setting. In some examples, the processor may perform (e.g., process) time-insensitive data processing operations based on a time window setting. The data communication may comprise one or more of physiological data and/or care usage data communicated to external circuitry.

As shown in association with at least FIG. 4B, examples are not limited to IMDs that include an acceleration sensor 110 of FIG. 4A. FIG. 4B is a block diagram schematically representing an example sensor type 130. In some examples, sensor type 130 corresponds to a sensor (e.g., 25 in FIG. 2) and/or a sensing function. As shown in association with at least FIG. 4B, sensor type 130 comprises various types of sensor modalities 131-144, any one of which may be used for determining, obtaining, and/or identifying care usage patterns, respiratory information, cardiac information, sleep quality information, activity patterns, sleep disordered breathing-related information, and/or other information related to providing patient care.

In some examples, sensor type 130 comprises the modalities of pressure 144, impedance 135, acceleration 143, airflow 136, radio frequency (RF) 138, optical 132, electromyography (EMG) 139, electrocardiography (ECG) 140, ultrasonic 133, acoustic 141, image 137, internal electronics 142 and/or other 134. In some examples, sensor type 130 comprises a combination of at least some of the various sensor modalities 131-144.

It will be understood that, depending upon the attribute being sensed, in some instances a given sensor modality identified within FIG. 4B may include multiple sensing components while in some instances, a given sensor modality may include a single sensing component. Moreover, in some instances, a given sensor modality identified within FIG. 4B may include power circuitry, monitoring circuitry, and/or communication circuitry and/or other internal electronics 142. However, in some instances a given sensor modality in FIG. 4B may omit some power, monitoring, and/or communication circuitry but may cooperate with such monitoring or communication circuitry located elsewhere.

In some examples, a pressure sensor 144 may sense pressure associated with respiration and may be implemented as an external sensor and/or an implantable sensor. In some instances, such pressures may include an extrapleural pressure, intrapleural pressures, etc. For example, one pressure sensor 144 may comprise an implantable respiratory sensor, such as that disclosed in U.S. Patent Publication No. 2011/0152706, published on Jun. 23, 2011, entitled “METHOD AND APPARATUS FOR SENSING RESPIRATORY PRESSURE IN AN IMPLANTABLE STIMULATION SYSTEM”, the entire teachings of which is incorporated herein by reference in its entirety.

In some examples, a pressure sensor 144 may sense sound and/or pressure waves at a different frequency than occur for respiration (e.g., inspiration, exhalation, etc.). In some instances, this data may be used to track cardiac parameters of patients via a respiratory rate and/or a heart rate. In some instances, such data may be used to approximate electrocardiogram information, such as a QRS complex. In some instances, the detected heart rate is used to identify a relative degree of organized heart rate variability, in which organized heart rate variability may enable detecting apneas or other sleep disordered breathing events, which may enable evaluating efficacy of sleep disordered breathing.

In some examples, pressure sensor 144 comprises piezoelectric element(s) and may be used to detect sleep disordered breathing (SDB) events (e.g., apnea-hypopnea events), to detect onset of inspiration, and/or detection of an inspiratory rate, etc. However, examples are not so limited and may comprise of variety of different types of IMDs.

As shown in FIG. 4B, in some examples one sensor modality includes airflow sensor 136, which may be used to sense respiratory information, sleep disordered breathing-related information, sleep quality information, etc. In some instances, airflow sensor 136 detects a rate or volume of upper respiratory air flow.

As shown in FIG. 4B, in some examples one sensor modality includes impedance sensor 135. In some examples, impedance sensor 135 may be implemented via various sensors distributed about the upper body for measuring a bio-impedance signal, whether the sensors are internal and/or external. In some examples, the impedance sensor 135 senses an impedance indicative of an upper airway collapse.

In some instances, the sensors are positioned about a chest region to measure a trans-thoracic bio-impedance to produce at least a respiratory waveform.

In some instances, at least one sensor involved in measuring bio-impedance may form part of a pulse generator, whether implantable or external. In some instances, at least one sensor involved in measuring bio-impedance may form part of a stimulation element and/or stimulation circuitry. In some instances, at least one sensor forms part of a lead extending between a pulse generator and a stimulation element.

In some examples, impedance sensor 135 is implemented via a pair of elements on opposite sides of an upper airway. Some example implementations of such an arrangement are further described herein.

In some examples, impedance sensor 135 may take the form of electrical components not used in an IMD. For instance, some patients may already have a cardiac care device (e.g., pacemaker, defibrillator, etc.) implanted within their bodies, and therefore have some cardiac leads implanted within their body. Accordingly, the cardiac leads may function together or in cooperation with other resistive/electrical elements to provide impedance sensing.

In some examples, whether internal and/or external, impedance sensor(s) 135 may be used to sense an ECG signal.

In some examples, impedance sensor 135 is used to detect SDB events (e.g., apnea-hypopnea events), to detect onset of inspiration, and/or detection of an inspiratory rate, etc.

As shown in FIG. 4B, in some examples one sensor modality includes an acceleration sensor 143. In some instances, acceleration sensor 143 is generally incorporated within or associated with the IMD. For instance, in some examples of an IMD, a housing (e.g., can) contains numerous components such as control circuitry, stimulation, and also may contain the acceleration sensor 143 within the housing. However, in some examples, the acceleration sensor 143 may be separate from, and independent of, the IMD. In some examples, acceleration sensor 143 may enable sensing body position, posture, and/or body motion regarding the patient, which may be indicative of patient behavior (e.g., activity pattern) and/or states, from which a care cycle may be selected. Among other uses, the data obtained via the acceleration sensor 143 may be employed to adjust a care usage pattern used to set the data event parameter of the IMD.

In some examples, acceleration sensor 143 enables acoustic detection of cardiac information, such as heart rate via motion of tissue in the head/neck region, similar to ballistocardiogram and/or seismocardiogram techniques. In some examples, measuring the heart rate includes sensing heart rate variability. In some examples, acceleration sensor 143 may sense respiratory information, such as but not limited to, a respiratory rate. In some examples, whether sensed via an acceleration sensor 143 alone or in conjunction with other sensors, one may track cardiac information and respiratory information simultaneously by exploiting the behavior of the cardiac signal in which a cardiac waveform may vary with respiration.

In some examples, acceleration sensor 143 is used to detect SDB events (e.g., apnea-hypopnea events), to detect onset of inspiration, and/or detection of an inspiratory rate, etc. In some examples, the acceleration sensor 143 may be used to detect SDB events during the sleep period and/or may be used continuously to detect arrhythmias. In some examples, the acceleration sensor 143, detection of cardiac information, and/or detection of SDB events may be implemented as described within U.S. Patent Publication No. 2019/0160282, published on May 30, 2019, entitled “ACCELEROMETER-BASED SENSING FOR SLEEP DISORDED BREATHING (SDB) CARE”, and/or U.S. patent application Ser. No. 16/977,664 filed on Sep. 2, 2020, and entitled “RESPIRATION DETECTION”, the entire teachings of which are each incorporated herein by reference in their entirety.

In some examples, RF sensor 138 shown in FIG. 4B enables non-contact sensing of various additional physiologic parameters and information, such as but not limited to respiratory information, cardiac information, motion/activity, and/or sleep quality. In some examples, RF sensor 138 enables non-contact sensing of physiologic data. In some examples, RF sensor 138 determines chest motion based on Doppler principles.

In some examples, one sensor modality may comprise an optical sensor 132 as shown in FIG. 4B. In some instances, optical sensor 132 may be an implantable sensor and/or external sensor. For instance, one implementation of an optical sensor 132 comprises an external optical sensor for sensing heart rate and/or oxygen saturation via pulse oximetry. In some instances, the optical sensor 132 enables measuring oxygen desaturation index (ODI).

As shown in FIG. 4B, in some examples one sensor modality comprises an EMG sensor 139, which records and evaluates electrical activity produced by muscles, whether the muscles are activated electrically or neurologically. In some instances, the EMG sensor 139 is used to sense respiratory information, such as but not limited to, respiratory rate, apnea events, hypopnea events, whether the apnea is obstructive or central in origin, etc. For instance, central apneas may show no respiratory EMG effort. In some examples, EMG sensing may be used to determine upper airway patency. In some such examples, the upper airway patency may be inferred and/or determined via EMG sensing based on sensing a degree of tongue protrusion, degree of tongue muscle contraction, degree of contraction of other upper airway patency-related muscles and the like.

In some instances, the EMG sensor 139 may comprise a surface EMG sensor while, in some instances, the EMG sensor 139 may comprise an intramuscular sensor. In some instances, at least a portion of the EMG sensor 139 is implantable within the patient's body and therefore remains available for performing electromyography on a long term basis.

In some examples, one sensor modality may comprise ECG sensor 140 which produces an ECG signal. In some instances, the ECG sensor 140 comprises a plurality of electrodes distributable about a chest region of the patient and from which the ECG signal is obtainable. In some instances, a dedicated ECG sensor(s) 140 is not employed, but other sensors such as an array of impedance sensors 135 (e.g., bio-impedance sensors) are employed to obtain an ECG signal. In some instances, a dedicated ECG sensor(s) is not employed but ECG information is derived from a respiratory waveform, which may be obtained via any one or several of the sensor modalities in sensor type 130 in FIG. 4B.

In some examples, an ECG signal obtained via ECG sensor 140 may be combined with respiratory sensing (via pressure sensor 144 or impedance sensor 135) to determine minute ventilation, as well as a rate and phase of respiration. In some examples, the ECG sensor 140 may be exploited to obtain respiratory information.

In some examples, ECG sensor 140 is used to detect SDB events (e.g., apnea-hypopnea events), to detect onset of inspiration, and/or detection of an inspiratory rate, etc.

As shown in FIG. 4B, in some examples one sensor modality includes an ultrasonic sensor 133. In some instances, ultrasonic sensor 133 is locatable in close proximity to an opening (e.g., nose, mouth) of the patient's upper airway and via ultrasonic signal detection and processing, may sense exhaled air to enable determining respiratory information, sleep quality information, sleep disordered breathing information, etc.

In some examples, acoustic sensor 141 comprises piezoelectric element(s), which sense acoustic vibration. In some implementations, such acoustic vibratory sensing may be used to detect sounds associated with SDB events (e.g., apnea-hypopnea events), to detect onset of inspiration, and/or detection of an inspiratory rate, etc.

In some examples, data via sensor types 130 in FIG. 4B, such as but not limited to physiological data, may be used in a training mode of the IMD to correlate various patterns in the sensed information with expected, observed and/or current care cycles.

As may be appreciated, examples are not limited to the implantable sensors and/or combinations as illustrated in associated with at least FIGS. 4A-4B and may include a variety of different implantable sensors, combinations, and other circuitry.

In some examples of the present disclosure, an IMD and/or IMD system may include multiple implantable sensors. In some examples, one or more of the implantable sensors may be separate from the respective IMD.

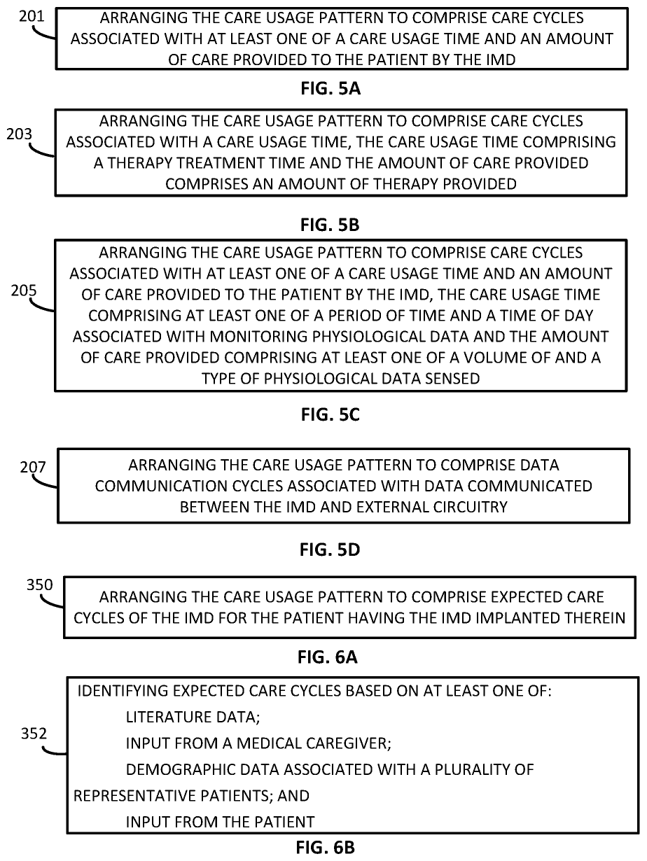

FIGS. 5A-8D are diagrams, which may comprise part of and/or are example implementations of example methods. In some examples, the methods illustrated by FIGS. 5A-8D may comprise part of and/or are example implementations of the method 10 illustrated by FIG. 1.

As shown at 201 in FIG. 5A, one example implementation comprises arranging the care usage pattern to comprise care cycles associated with at least one of a care usage time and an amount of care provided to the patient by the IMD. In some examples, care usage time, as used herein, comprises and/or refers to at least one of a time of day and a period of time care is provided to or anticipated as being provided to the patient. In some examples, as shown at 203 in FIG. 5B, the method comprises arranging the care usage pattern to comprise care cycles associated with the care usage time, the care usage time comprising a therapy treatment time and the amount of care provided comprising an amount of therapy provided. The amount of therapy may comprise an intensity of therapy provided. In some examples, the intensity of therapy may be adjusted at or based on a time of day, an activity type occurring, and an amount of activity. However, examples are not limited to an IMD providing therapy. In some examples, the care usage pattern may be indicative of or include cycles for monitoring the physiological data via the IMD, such as cycles of changed sensitivity for monitoring the physiological data. For example, as shown at 205 in FIG. 5C, the method comprises arranging the care usage pattern to comprise care cycles associated with at least one of the care usage time and an amount of care provided to the patient by the IMD. In some examples, the care usage time may comprise at least one of a period of time and a time of day associated with monitoring physiological data. In some examples, the amount of care provided may comprise at least one of a volume of and a type of physiological data sensed.

In some examples, as shown at 207 in FIG. 5D, the method comprises arranging the care usage pattern to comprise data communication cycles associated with data communicated between the IMD and external circuitry. For example, the method may further comprise predicting, based on the care usage pattern, a time of day the IMD is to communicate data with external circuitry, and setting the data event parameter based on the prediction.

FIGS. 6A-6B, FIGS. 7A-7E, and FIGS. 8A-8D illustrate example methods involving different care usage patterns, and may comprise part of and/or are example implementations of the method (e.g., 10) as illustrated in association with at least FIGS. 1 and 5A-8D.

As shown at 350 in FIG. 6A, the method comprises arranging the care usage pattern to comprise expected care cycles of the IMD for the patient having the IMD implanted therein. For example, at shown at 352 in FIG. 6B, the method comprises identifying the expected care cycles based on at least one of literature data, input from a medical caregiver, demographic data associated the patient and/or with a plurality of representative patients, and/or input from the patient. For example, the expected care cycles may comprise predicted times of providing care to the user, predicted times of not providing care to the user, and/or predicted times of data communication with external circuitry based on externally input data, such as data input by a medical caregiver to program the IMD for the patient.

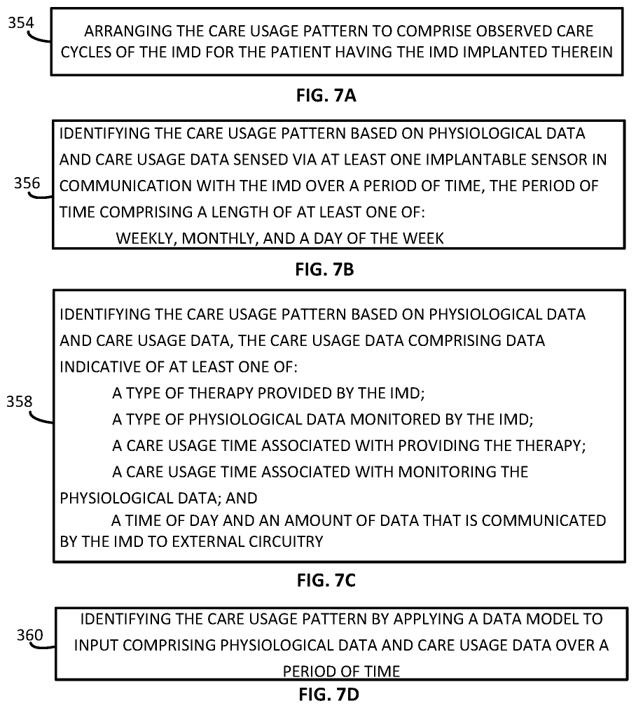

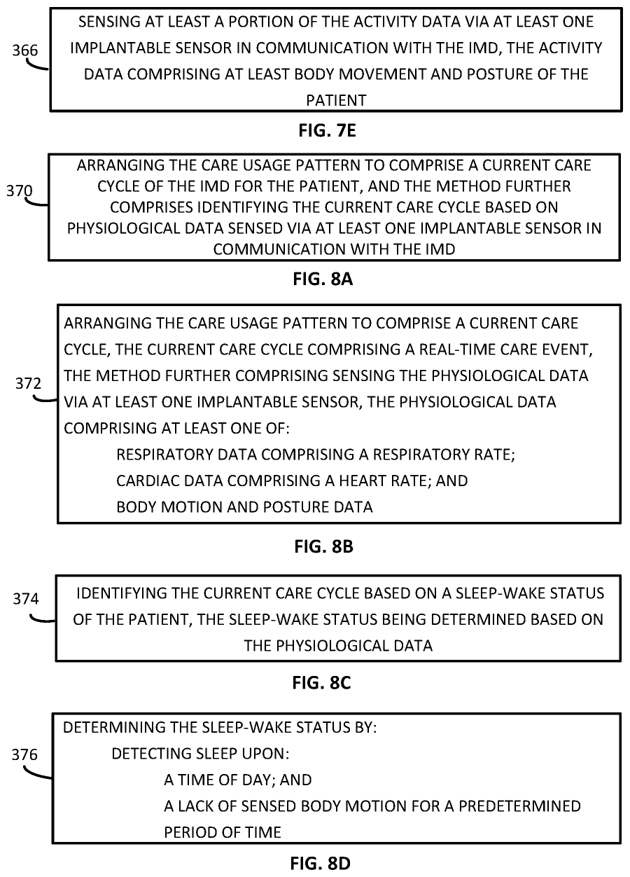

In some examples, as shown at 354 in FIG. 7A, the method comprises arranging the care usage pattern to comprise observed care cycles of the IMD for the patient having the IMD implanted therein. For example, the method of FIG. 7A, as shown at 356 in FIG. 7B, may further comprise identifying the care usage pattern based on physiological data and care usage data sensed via at least one implantable sensor in communication with the IMD over a period of time. The period of time may comprise a length of at least one of weekly, monthly, and a day of the week, however, examples are not so limited.

In some examples, as shown at 358 in FIG. 7C, the method comprises identifying the care usage pattern using care usage data, the care usage data comprising data indicative of at least one of: i) a type of therapy provided by the IMD, ii) a type of physiological data monitored by the IMD, a care usage time associated with providing the therapy, iii) a care usage time associated with monitoring the physiological data, and iv) a time of day and an amount of data (e.g., physiological data, portions of the care usage data, or other data) that is communicated by the IMD to external circuitry. The physiological data may be sensed by at least one implantable sensor in communication with the IMD. In some examples, the at least one implantable sensor may comprise at least some of substantially the same attributes and features as the implantable sensors as previously described in association with at least FIGS. 4A-4B. In some examples, the physiological data comprises bioimpedance, an electroencephalogram (EEG) signal, an IPG signal, respiratory data comprising a respiratory rate, cardiac data comprising a heart rate, body motion data, posture data, and various combinations thereof.

In some examples, as shown at 360 in FIG. 7D, the method comprises identifying the care usage pattern by applying a data model to input comprising physiological data and care usage data over a period of time. In some examples, the input to the data model comprises the set of factors, as described previously in association with at least FIG. 1. For example, the care usage data may comprise at least one of a type, an amount, a period of time, a time of day, and a day of the week associated with providing care by the IMD. In some examples, the input may comprise activity data associated with the patient. For example, at shown at 366 in FIG. 7E, the method may further comprise sensing at least a portion of the activity data via at least one implantable sensor in communication with the IMD, the activity data comprising at least body movement and posture of the patient. In some examples, the data model may be constructed and/or otherwise include at least some of substantially the same features and attributes as the constructed data model as further shown in association with at least FIGS. 24-29.

In some examples, as shown at 370 in FIG. 8A, the method comprises arranging the care usage pattern to comprise a current care cycle of the IMD for the patient, and the method (such as method 10 of FIG. 1) comprises identifying the current care cycle based on physiological data sensed via at least one implantable sensor in communication with the IMD. In some examples, the at least one implantable sensor may comprise at least some of substantially the same attributes and features as the implantable sensors previously described and associated with at least FIGS. 4A-4B. In some examples, as shown at 372 in FIG. 8B, the method comprises arranging the care usage pattern to comprise the current care cycle, the current care cycle comprising a real-time care event. The method may further comprise sensing the physiological data via the at least one implantable sensor in communication with the IMD. In some examples, the physiological data may comprise a respiratory data comprising a respiratory rate, cardiac data comprising a heart rate, and/or body motion and posture data.

In some examples, as shown at 374 in FIG. 8C, the method comprises identifying the current care cycle based on a sleep-wake status of the patient. For example, the sleep-wake status may be determined based on the physiological data. A sleep-wake status, as used herein, comprises and/or refers to a state of a patient that is indicative of the patient being asleep or awake. Some example methods described herein may comprise determining a sleep-wake status of a patient using at least some of the substantially the same features and attributes as described in U.S. patent application Ser. No. 16/978,470, filed Sep. 4, 2020, and entitled “SLEEP DETECTION FOR SLEEP DISORDERED BREATHING (SDB) CARE”, the entire teachings of which is incorporated herein by reference in its entirety.

In some examples, the method comprises identifying the care usage pattern based on the sleep-wake status of the patient. In some examples, the method further comprises determining the sleep-wake status based on at least one of: i) the time of day, ii) day of week, iii) a lack of sensed body motion for a predetermined period of time, iv) demographic data indicative of sleep schedules for representative patients, v) input data indicative of a sleep schedule for the patient and/or vi) physiological data. In some examples, the method comprises determining the sleep-wake status by detecting sleep upon a time of day and a lack of sensed body motion for a predetermined period of time, as shown at 376 in FIG. 8D.

As described above, in some examples, a sleep-wake status is determined based on respiration data. In some such examples, the method may comprise determining the respiration data based on sensing motion of a chest wall, and determining the sleep-wake status by differentiating, based on an amplitude of the sensed chest wall motion, between active respiration indicative of an awake state and passive respiration indicative of a sleep state.

In some examples, the sleep-wake status is determined by sensing variability in at least one of the respiratory rate, the heart rate, and body motion. In some such examples, performing the determination of the sleep-wake status may be based on the variability in at least one of the respective sensed respiratory rate, the heart rate, and the body motion. For example, the method may further comprise determining the sleep-wake status based on at least one of determining, from the cardiac data, a heart rate variability (HRV) and the heart rate.

In some examples, methods (e.g., 10) as illustrated in association with at least FIGS. 1 and 5A-8D, the method may include use of different combinations of the expected care cycles, the observed care cycles, and the current care cycle. For example, the IMD may be programmed initially with the expected care cycles. The IMD may set at least one data event parameter, such as a polling interval and/or time window for data processing, based on a particular one of the expected care cycles, which may be identified by at least one factor. Overtime, and based on physiological data and/or care usage data obtained by the IMD and/or at least one implantable sensor, the IMD may identify the observed care cycles and revise the at least one data event parameter based on at least one of the observed care cycles. In some examples, the IMD may be reprogrammed with and/or overwrites the expected care cycles with the observed care cycles. In some examples, the care usage pattern may comprise or be arranged as combinations of expected and observed care cycles.

In some examples, the expected care cycles and/or observed care cycles may be temporarily overridden by a current care cycle. For example, the IMD may provide care at a time that conflicts with the particular expected care cycle and/or observed care cycle. Based on the current care being provided, the data event parameter may be adjusted until care discontinues and/or in response to the care discontinuing. The current care cycle, if occurs repetitively, may be used to adjust the observed care cycles.

FIGS. 9A-9C are block diagrams schematically illustrating an example care usage engine 27 of an IMD system. In some examples, the care usage engine 27 and/or IMD system of FIGS. 9A-9C may implement the various methods described above, including but not limited to, the method (e.g., method 10) illustrated in association with FIG. 1 as well as FIGS. 5A-8D. As shown in association with at least FIG. 9A, the care usage engine 27 may include a plurality of sub-engines, e.g., the movement sub-engine 215, the care cycle sub-engine 240, the physiological data sub-engine 270, and the other input sub-engine 285. In some examples, the plurality of sub-engines may provide inputs used by the care usage engine 27 for identifying a care usage pattern and/or to set a data event parameter based on the care usage pattern. In some examples, the plurality or a sub-portion of the plurality of sub-engines may work independent of one another and/or cooperatively together to help determine the care usage pattern.

The care usage engine 27 may include a movement sub-engine 215 used to determine body motion data 220 and posture data 230. As previously described at least in connection with FIG. 4B, the body motion data 220 and posture data 230 may be determined from forces sensed from an acceleration sensor.

FIG. 9B illustrates an example of a movement sub-engine 215. As shown, the movement sub-engine 215 may be used to detect, determine or designate body motion data 220 and posture data 230 based upon the data sensed by the at least one implantable sensor. The body motion data 220 and posture data 230 may be indicative of a pattern of motion or movement of the patient. In some examples, the body motion data 220 and posture data 230 may be used to identify an activity pattern of the patient. For example, the body motion data 220 may comprise information related to the type of motion 222, the intensity of the motion 224, and the duration of the motion 226. The posture data 230 may similarly include the type of posture 232 and the duration of the posture 234. The movement sub-engine 215 may identify a pattern, such as an order of motion(s) and posture(s) 228 and which may be used to identify an activity pattern of the patient.

The movement sub-engine 215 may determine body motion data 220 of the patient, such as determining whether the patient is active or at rest. In some examples, when a vector magnitude of the acceleration measured via the acceleration sensor meets or exceeds a threshold (optionally for a period of time), the measurement may indicate the presence of non-gravitational components indicative of body movement. In some examples, the threshold is about 1.15G. Conversely, measurements of acceleration of about 1G (corresponding to the presence of the gravitational components only) may be indicative of rest.

The movement sub-engine 215 may determine posture data 230, including the type of posture 232, by determining whether at least an upper body portion (e.g., torso, head/neck) of the patient is in a generally vertical position (e.g., upright position) or lying down. In some examples, a generally vertical position may comprise standing or sitting.

In some examples, the movement sub-engine 215 determines the posture data 230 by rejecting non-posture components from an acceleration sensor via low pass filtering relative to each axis of the multiple axes of the acceleration sensor. In some examples, posture is at least partially determined via detecting a gravity vector from the filtered axes.

In some examples, if the measured angle is greater than a threshold (e.g., 40) degrees, then the measured angle indicates that the patient is lying down. In some such examples, a posture classification implemented by the movement sub-engine 215 includes classifying sub-postures, such as whether the patient is in a supine position, a prone position, or in a lateral decubitus position. In some non-limiting examples, after confirming a likely position of lying down, the movement sub-engine 215 determines if the patient is in a supine position or a prone position. However, examples are not so limited and the patient position or posture may be determined using other techniques, such as use of a dot product of the vectors.

In some examples, the movement sub-engine 215 is programmed to distinguish between a supine sleep position and a generally supine reclined position. As a point of reference, a generally supine reclined position may be one in which the patient is on a recliner, on an adjustable-type bed, laying on a couch, or the like and not attempting to sleep (e.g., watching television) as compared to sleeping in bed. An absolute vertical distance between the head and torso of the patient in the supine sleep position is less than the absolute vertical distance between the head and torso in the generally supine reclined position.

In some examples, the movement sub-engine 215 is programmed to consider or characterize a position of the patient's neck and/or head and/or body position (e.g., as part of a determination of the patient's rotational position while lying down). For example, the movement sub-engine 215 is programmed to estimate a position of the patient's neck based on body position. A determination that the patient's torso is slightly offset may imply different head positions. In some non-limiting examples, the systems and methods of the present disclosure may consider or characterize a position of the patient's neck and/or head via information from a sensor provided with a microstimulator that is implanted in the patient's neck or in conjunction with a sensor integrated into the stimulation lead. In some examples, two (or more) acceleration sensors may be provided, each implanted in a different region of the patient's body (e.g., torso, head, neck) and providing information to the movement sub-engine 215 sufficient to estimate neck and/or head and/or body positions of the patient.

The above explanations provide a few non-limiting examples of some posture determination or designation protocols implemented by the movement sub-engine 215. However, examples are not so limited and a number of other posture determination or designation techniques are also envisioned by the present disclosure, and may be function of the format of the implantable sensor and/or other information provided by one or more additional sensors. Various body postures and sub-postures may be determined or designated as implemented and described within U.S. patent application Ser. No. 16/978,275, filed Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety.

Some systems and methods of the present disclosure may comprise calibrating data sensed to compensate, account, or address the possibility that a position of the at least one implantable sensor (from which posture determinations may be made) within the patient's body is unknown and/or has changed over time. In some examples, the calibration may be based on establishing a horizontal baseline gravity plane, establishing or creating a vertical baseline gravity vector and a horizontal baseline gravity plane, and/or receiving a predetermined vertical baseline gravity vector and one or more predetermined horizontal baseline gravity vectors, based upon respiratory and/or cardiac waveform polarity information provided by or derived from the implantable sensor, among other variations as described within U.S. patent application Ser. No. 16/978,275, filed Sep. 4, 2020, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety.

Referring back to FIG. 9A, in some examples, the care usage engine 27 further includes a care cycle sub-engine 240. The care cycle sub-engine 240 may identify an expected care cycle 250, an observed care cycle 260 and/or a current care cycle 267, as described above.

FIG. 9C illustrates an example of a care cycle sub-engine 240. As shown, the care cycle sub-engine 240 may be used to detect, determine or identify expected care cycle data 250, observed care cycle data 260, and/or a current care cycle data 267 based on the data sensed by the at least one implantable sensor. The expected care cycle data 250, observed care cycle data 260, and current care cycle data 267 may be indicative of a pattern of care provided by the IMD, and optionally, in some examples, indicative of times of the day the IMD communications or is anticipated to communicate with external circuitry. For example, the expected care cycle data 250 may include data from literature 252, data input from a medical caregiver 256, demographic data 258 related to the patient and/or representative patients, among other data 254. The observed care cycle data 260 may include care usage data 268 and physiological data 280. As shown, the care usage data 268 may include data indicative of a type of care provided 273, an intensity of the care provided 269, a duration of the care provided 271, a time 272, and an order of care provided 265. In some examples, the time 272 may include a time of day the care is provided, a period of time the care is provided, among other information, such as the day of the week associated with the care. The current care cycle data 267 may include data such as the physiological data 262 and the sleep-wake status 264, among other data in some examples. The care cycle sub-engine 240 may identify a particular care cycle (e.g., expected, observed or current care cycle) of the care usage pattern based on the expected care cycle data 250, observed care cycle data 260, and/or current care cycle data 267 (e.g., set of factors), and set at least one data event parameter based on the particular care cycle.

Referring back to FIG. 9A, the care usage engine 27 may further include a physiological data sub-engine 270. The physiological data sub-engine 270 may collect physiological data, such as cardiac data 275 and/or respiratory data 281, among other data in some examples.

In some examples, the care usage engine 27 may further include other sub-engines, as illustrated by the other inputs sub-engine 285. The other inputs sub-engine 285 may include one or more engines which are used to determine different inputs to the care usage engine 27. The other inputs may include a temporal parameter, such as the time of the day 286, time of the year 287, time zone 288, and/or patterns of activity 289. In some examples, the other inputs may include a subset of the set of factors, as previously described in connection with at least FIG. 1.

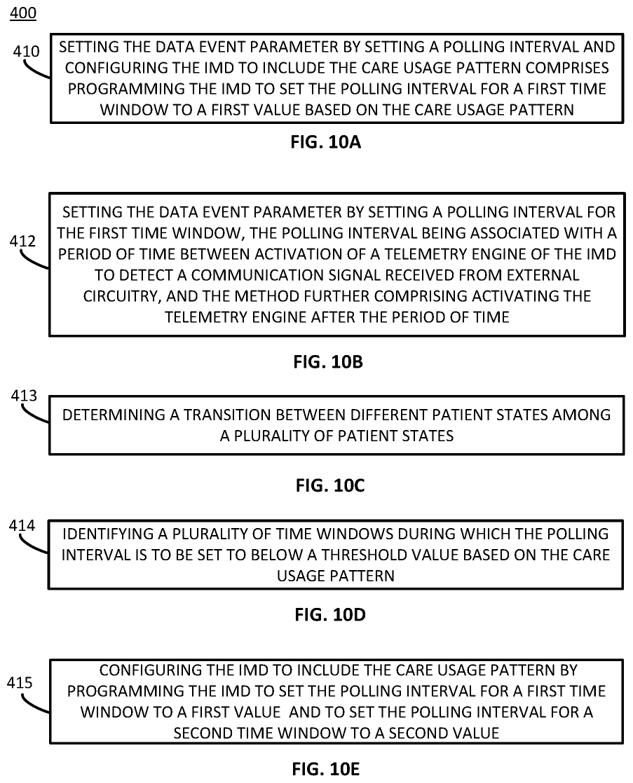

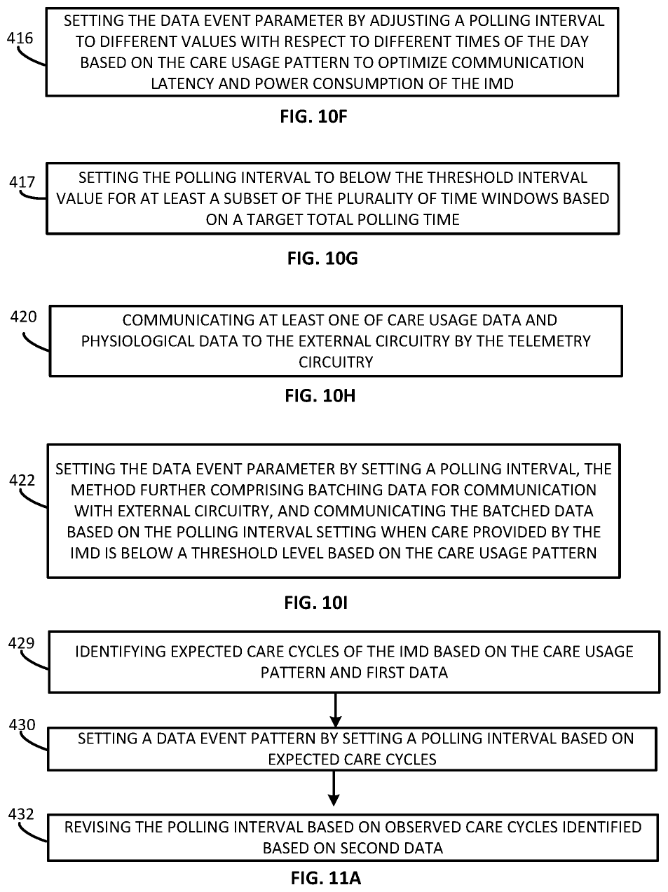

FIGS. 10A-13B are diagrams, which may comprise part of and/or are example implementations of example methods. In some examples, the methods illustrated by FIGS. 10A-13B may comprise part of and/or are example implementations of the method (e.g., 10) illustrated in association with at least FIGS. 1 and 5A-8D.

As described above, in some examples, the data event parameter comprises a polling interval. Setting the polling interval based on a care usage pattern of the IMD may be used to balance communication latency with power performance of the IMD. In some example methods, such as method 400 as shown at 410 in FIG. 10A, the method comprises setting the data event parameter by setting a polling interval, and configuring the IMD to include the care use pattern comprises programming the IMD to set the polling interval for a first time window to a first value based on the care usage pattern. In some examples, setting the polling interval for the first time window to the first value may be used to optimize power performance and/or energy consumption performance of the IMD. In some examples, the method comprises setting the data event parameter by setting a polling interval for the first time window to the first value, and as shown at 412 in FIG. 10B, the polling may be associated with a period of time between activation of telemetry circuitry of the IMD to detect a communication signal received from external circuitry. In some examples, the method may further comprise activating the telemetry circuitry after the period of time (e.g., after each).

In some examples, the polling interval is set based on a time of day. In some examples, the polling interval is set based on at least one patient state, such as a sleep-wake state of the patient. In some examples, shown at 413 in FIG. 10C, the method 400 of FIG. 10A may further comprise determining a transition between different patient states among a plurality of patient states. The different patient states may comprise at least one of different body positions (e.g., a laying state to a sitting state, a standing state to a laying state, etc.) and different sleep-wake statuses (e.g., sleeping state to an awake state), among other patient states and various combinations thereof.

In some examples, as shown at 414 in FIG. 10D, the method 400 of FIG. 10A further comprises identifying a plurality of time windows during which the polling interval is to be set to below a threshold value based on the care usage pattern. For example, after setting the polling interval, as shown at 415 in FIG. 10E, the method 400 may comprise configuring the IMD to include the care usage pattern by programming the IMD to set the polling interval for a first time window to a first value and to set the polling interval for a second time window to a second value, where the second value is greater than the first value. For example, as shown at 416 in FIG. 10F, the method may comprise setting the data event parameter by adjusting a polling interval to different values with respect to different times of the day based on the care usage pattern to optimize communication latency and energy consumption and/or power performance of the IMD.

In some examples, as shown at 417 in FIG. 10G, the method 400 comprises setting the polling interval to below a threshold interval value for at least a subset of the plurality of time windows based on a target total polling time. For example, the method may comprise identifying a plurality of time windows based on the care usage pattern, and setting the polling interval below the threshold interval value for at least the subset of the plurality of time windows may be used to limit a total amount of time in a day that the IMD is set to the polling interval below the threshold interval value. In some examples, setting the polling interval for the subset of the plurality time windows based on the target total polling time may be used to limit a total daily polling time. For example, the total daily polling time may be limited to a maximum of the target total polling time (e.g., a maximum value for the polling time is the target total polling time) and allocating the total daily polling time based on the care usage pattern. As an example, if the target total polling time is one hour, the totally daily polling time for the IMD may be limited to one hour or less and which may be allocated over the subset of or the plurality of time windows. In some such examples, the care usage pattern may include observed communication patterns of the IMD (e.g., user connects to IMD upon waking up, when sitting at 9 pm, etc.).

In some examples, the method 400 of FIG. 10A further comprises communicating at least one of care usage data and physiological data to the external circuitry by the telemetry circuitry, as shown at 420 in FIG. 10H. The data may be communicated based on the data event parameter setting.

In some examples, the method 400 may further comprise batching data based on the set polling interval. For example, as shown at 422 in FIG. 10I, the method may comprise setting the data event parameter by setting a polling interval, batching data for communication with external circuitry, and communicating the batched data based on the polling interval setting when care provided by the IMD is below a threshold level based on the care usage pattern. The data that is batched for communication may include at least one of care usage data and physiological data.

In some examples, the batched data may be communicated in response to a battery parameter being within a threshold level (e.g., a battery power delivering capacity, such as 2.5V) and based on the data event parameter. In some examples, the battery parameter may include a battery capacity (e.g., how much the battery is being utilized), and if the battery capacity is at or below the threshold level (e.g., battery is not being fully utilized), the batched data may be communicated. In other examples, the battery parameter may include a battery voltage, battery power delivery capacity, available power and/or used power. For example, the method 400 may further comprise identifying that care provided by the IMD is outside a threshold level, and in response, batching the data for subsequent data communication. In some examples, the method 400 may further comprise communicating the batched data to external circuitry in response to the care provided being within the threshold level and based on the set data event parameter.

In some examples, the battery parameter may include or be associated with an amount of power remaining on a battery of the IMD and/or a predicted recharge time, such as with a rechargeable battery. For example, in response to the remaining battery power being below a threshold, it may be predicted that a recharge of the battery is to occur at a particular time (e.g., within an hour) and the batched data may be communicated after the recharge. In some examples, the recharge time may be predicted based on past patterns of recharge, such as the user recharging the battery at specific times and/or days of the week. In response, the data may be batched for subsequent data communication after the recharge. In other examples and/or in addition, in response to the amount of battery power remaining being below another (lower) threshold, an alert message may be communicated to notify the user to recharge the rechargeable battery or to notify that the primary battery is near depleted.

In some examples, the care usage pattern (e.g., expected, observed or current care cycles) may be identified by assessing one or more of: i) a probability of care being provided by the IMD, ii) a probability of communication occurring between the IMD and external circuitry, and iii) a probability of a battery parameter of the IMD being above or below a threshold level at a particular time of the day based on a pattern within external data (e.g., time of day and/or input expected care cycles) and/or internally-obtained data (e.g., physiological data, care usage data). Similarly, in some examples, the data event parameter may be set based on the prediction of when communication is likely to occur with external circuitry, the prediction of when care is likely to be provided by the IMD, and/or the prediction of the battery parameter being within the threshold level (e.g., a power threshold). The prediction(s) may include or be associated with a time of day, and the data event parameter is set based on the prediction. In some examples, the prediction(s) may be overruled or overridden by real-time detection of care being provided and/or battery parameter being outside the threshold level.

In some examples, the care usage pattern may include different cycles of care depending on activity of the user during the day (and/or other period of time), time of the day, day of the week, patterns of motion and/or posture, and various other inputs. As an example, a patient that has an activity level above a threshold level for the previous week, which is indicative of exercise, may have a different expected and/or observed care cycle than when they have an activity level below the threshold level for the previous week. As may be appreciated, motion patterns may include an identified lack of motion. As another example, a patient may have a different expected and/or observed care cycle during a work day than during the weekend and/or when on vacation. Such differences in care cycles may be expected initially and/or updated over time based on the observed care provided by the IMD, and which may change over time. As may be appreciated, the above examples are non-limiting and non-exclusive examples.

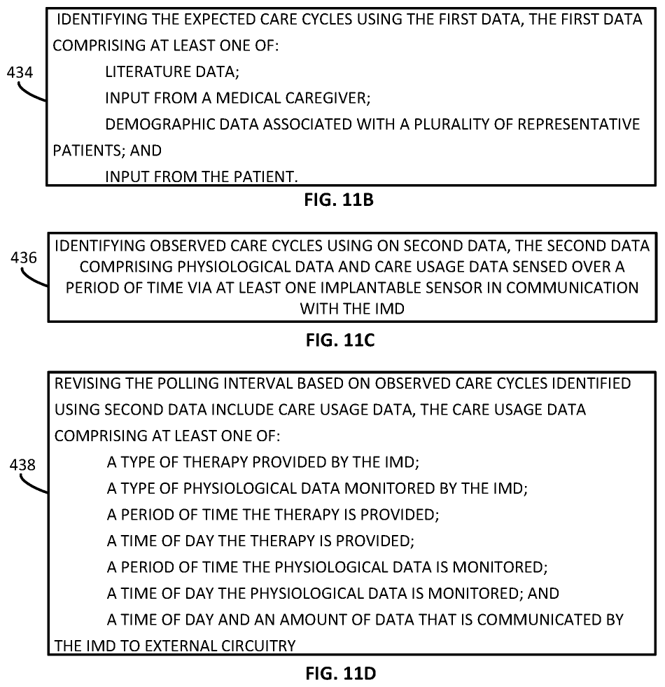

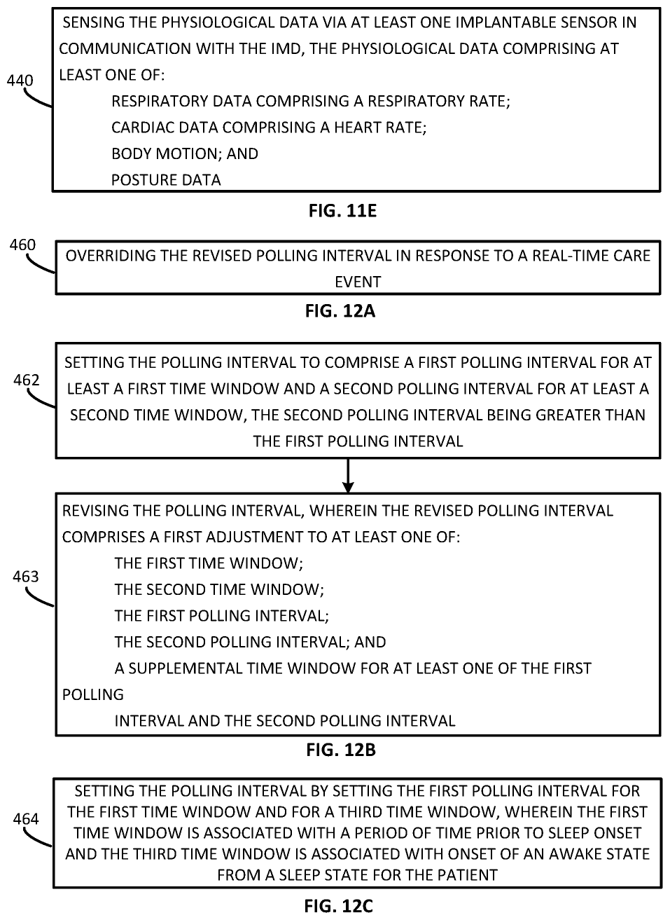

The methods illustrated by FIGS. 11A-11E may comprise part of and/or are example implementations of the method (e.g., 10) in association with at least FIG. 1 and FIGS. 5A-8D, and/or the method (e.g., 400) illustrated in association with at least FIGS. 10A-13B. In some example methods, the IMD may transition from using expected care cycles to using observed care cycles. For example, as shown at 429 and 430 in FIG. 11A, the method may comprise identifying expected care cycles of the IMD based on the care usage pattern and first data, and setting the data event parameter by setting a polling interval based on the expected care cycles. As shown at 432 in FIG. 11A the method 400 may further comprise revising the polling interval based on observed care cycles identified based on second data. In some examples, the first data may comprise data obtained from sources external to the IMD, and the second data may comprise data internally obtained by the IMD and/or from the at least one implantable sensor.

In some examples, as shown at 434 in FIG. 11B, the method may comprise identifying the expected care cycles using the first data. Non-limiting examples of first data includes literature data, input from a medical caregiver, demographic data associated with the patient and/or a plurality of representative patients, and input from the patient, as shown at 434 in FIG. 11B. In some examples, as shown at 436 in FIG. 11C, the method may comprise identifying the observed care cycles using the second data. Non-limiting examples of second data includes physiological data and care usage data sensed over a period of time via at least one implantable sensor in communication with the IMD, as shown at 436 in FIG. 11C.

As shown at 438 in FIG. 11D, in some examples, the method comprises revising the polling interval based on the observed care cycles identified using the second data that includes care usage data. In some examples, the care usage data includes at least one of: i) a type of therapy provided by the IMD, ii) a type of physiological data monitored by the IMD, iii) a period of time the therapy is provided, iv) a time of day the therapy is provided, v) a period of time the physiological data is monitored, a time of day the physiological data is monitored, vi) a time of day and an amount of data that is communicated by the IMD to external circuitry. As shown at 440 in FIG. 11E, a method, such as method 400 of FIG. 10A, may further comprise sensing the physiological data via at least one implantable sensor in communication with the IMD. Non-limiting examples of physiological data includes respiratory data comprising a respiratory rate, cardiac data comprising a heart rate body motion data, and posture data.

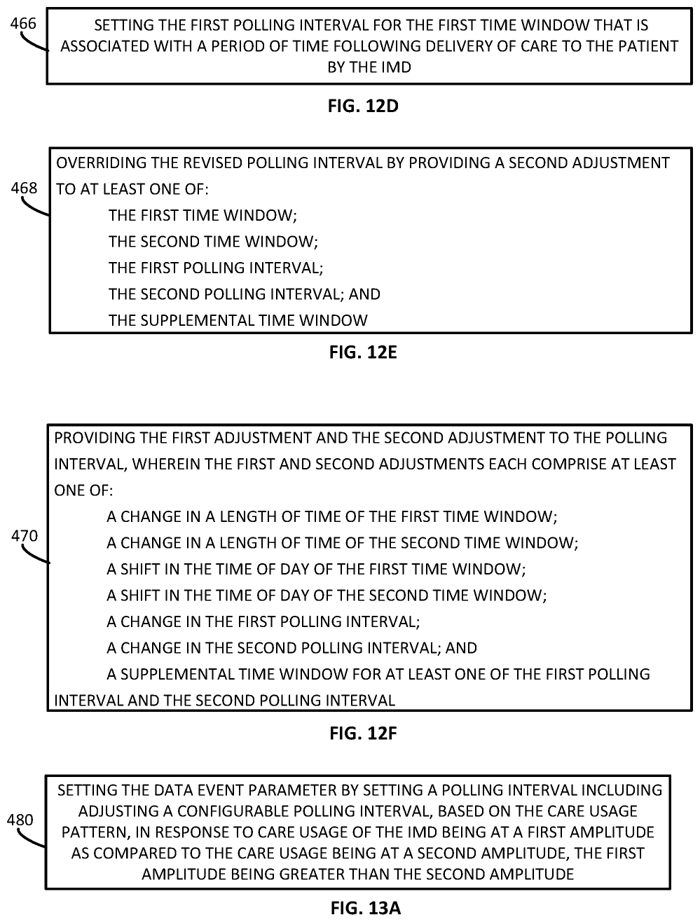

The methods illustrated by FIGS. 12A-12F may comprise part of and/or are example implementations of the method (e.g., 10) illustrated in association with FIG. 1 and FIGS. 5A-8D and/or the method (e.g., 400) illustrated in association with at least FIGS. 10A-13B. In some examples, the IMD may transition from expected or observed care cycles to using a current care cycle. For example, as shown at 460 in FIG. 12A, the method, such as method 400 of FIG. 10A, may comprise overriding the revised polling interval in response to a real-time care event. In some examples, the method comprises determining a current care cycle of the IMD based on physiological data sensed by the IMD, the current care cycle being associated with the real-time care event. In some examples, the revised polling interval may be overridden (e.g., no data communication or listening event) and/or a polling interval setting may be adjusted. For example, the polling interval may be adjusted to above a threshold value to reduce communication and/or a time window associated with the revised polling interval may be adjusted. In some examples, the revised polling interval may be based on the expected or observed care cycle, and may be adjusted based on a current care cycle (e.g., real time event).

As a particular example, a polling interval may initially be set based on expected care cycles. For example, communication between the IMD and external circuitry may be expected for a particular patient (and no care may be expected to be provided to the patient by the IMD) at a first time window. The IMD may have a first polling interval setting for the first time window (e.g., 8 am to 9 am) of 5 seconds. Outside the first time window, the IMD may have a second polling interval setting that is above a threshold value, such as 10 minutes. Overtime, observed care cycles of the IMD may indicate that the first time window can be revised (e.g., shortened) to a second time window of 8 am to 8:30 am. For example, communication between the IMD and external circuitry may be observed to occur between 8 am and 8:30 am, and observed to not occur after 8:30 am to 9 am as expected. The IMD may revise the first polling interval setting for the second time window of 8 am to 8:30 am and the second polling interval for times of the day outside the second time window. At 8:20 am on a particular day, care may be provided to the patient by the IMD, indicating a real time care event is occurring during the second time window and when the IMD is using the first polling interval. In response to the care being provided, the IMD may override the first polling interval (e.g., 5 second polling interval) by preventing communication with the IMD and/or preventing a listening event. In some examples, the IMD may additionally and/or alternatively adjust the first polling interval during the second time window to the second polling interval (e.g., to 10 minutes until the care ends). In some examples, the IMD may prevent communication with external circuitry until after the care ends and the IMD may adjust the polling interval for a respective time window after the care ends to the first polling interval. For example, communication between the IMD and external circuitry may be expected after care is provided. After the care stops, the IMD may adjust the polling interval for a third time window (e.g., 20-30 minutes) to the first polling interval. The third time window may overlap with the second time window (e.g., 8:25 am to 9:05 am) or be outside the second time window (e.g., 8:40 am to 9:10 am), in some examples. After the third time window lapses, the IMD may revert back to the second polling interval and/or other polling intervals based on observed care cycles and/or expected care cycles.

In some examples, the IMD may set multiple polling intervals and/or the polling interval may be set for multiple time windows. For example, as shown at 462 in FIG. 12B, in some examples of the method 400 of FIG. 12A, the method comprises setting the polling interval to comprise a first polling interval for at least a first time window and a second polling interval for at least a second time window, the second polling interval being greater than the first polling interval. In some such examples, as shown at 463 in FIG. 12B, the method comprises revising the polling interval (such as based on real time data and/or observed care cycles), wherein the revised polling interval may comprise: i) a first adjustment to at least one of the first time window, ii) the second time window, iii) the first polling interval, iv) the second polling interval, and v) a supplemental time window for at least one of the first polling interval and the second polling interval.

In some examples, as shown at 464 in FIG. 12C, the method may further comprise setting the polling interval by setting the first polling interval for the first time window and for a third time window, wherein the first time window is associated with a period of time prior to sleep onset and the third time window is associated with onset of an awake state from a sleep state for the patient, however examples are not so limited.

In some examples, as illustrated at 466 in FIG. 12D, the method comprising setting the first polling interval for the first time window that is associated with a period of time following delivery of care to the patient by the IMD. However, examples are not so limited, and in some examples the first time window may be associated with a period of time following a transition from a sleep state to an awake state for the patient, among other times.

As shown at 468 in FIG. 12E, in some examples, the method comprises overriding the revised polling interval by providing a second adjustment to at least one of the first time window, the second time window, the first polling interval, the second polling interval, and the supplemental time window. For example, as shown at 470 in FIG. 12F, the method comprises providing the first adjustment and the second adjustment to the polling interval, wherein the first and second adjustments each comprise at least one of: i) a change in a length of time of the first time window, ii) a change in a length of time of the second time window, iii) a shift in the time of day of the first time window, iv) a shift in the time of day of the second time window, iv) a change in the first polling interval; v) a change in the second polling interval; vi) a supplemental time window for at least one of the first polling interval and the second polling interval.

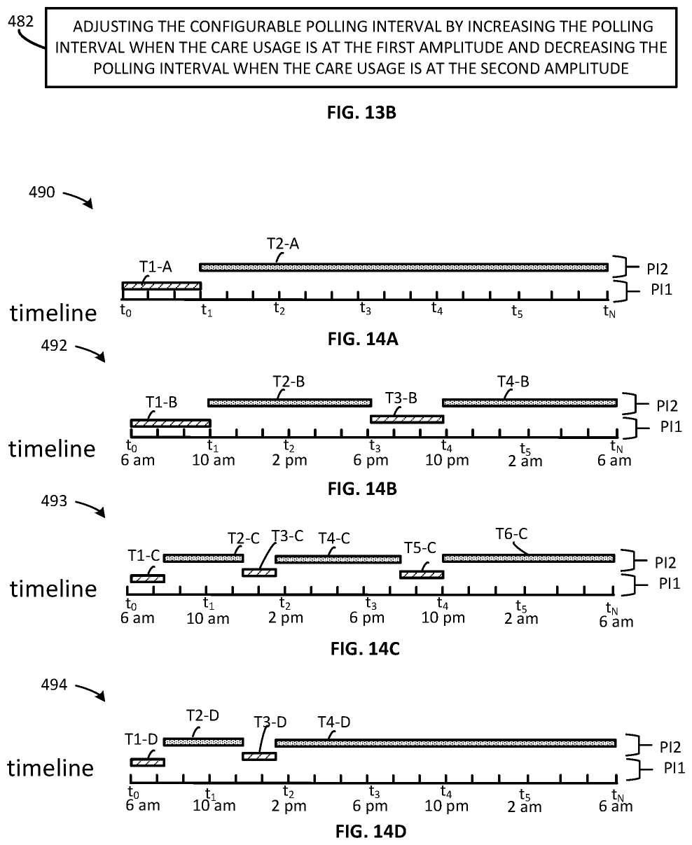

FIGS. 13A-13B illustrates further variations on methods for setting a polling interval. In some examples, the methods illustrated by FIGS. 13A-13B may comprise part of and/or are example implementations of the example method (e.g., 10) illustrated in association with at least FIG. 1 and FIGS. 5A-8D and/or the method 400 illustrated in association with at least FIGS. 10A-13B. As shown at 480 in FIG. 13A, the method may comprise setting the data event parameter by setting a polling interval including adjusting a configurable polling interval based on the care usage pattern and in response to care usage of the IMD being at a first amplitude as compared to the care usage being at a second amplitude. The first amplitude may be greater than the second amplitude.

In some examples, as shown at 482 in FIG. 13B, the method comprises adjusting the configurable polling interval by increasing the polling interval when care usage is at the first amplitude and decreasing the polling interval when care usage is at the second amplitude. In some examples, the care usage may be at the first amplitude in response to the IMD providing at least one of therapy and care to the patient that is above a threshold level to the patient. In some examples, adjusting the configurable polling interval comprises decreasing the polling interval when the care usage is at the first amplitude and/or where the care usage is at the first amplitude in response to the care provided to the patient being above a threshold level.

FIGS. 14A-14D are graphs illustrating example configurable polling intervals. In some examples, FIGS. 14A-14D may form part of or are example implementations of various example methods described herein, including but not limited method (e.g., 10) illustrated in associated with at least FIG. 1 and method 400 illustrated in association with at least FIGS. 10A-13B. FIG. 14A illustrates a generic timeline 490 showing a first time window (T1-A) associated with a first polling interval (PI1) and a second time window (T2-A) associated with a second polling interval (PI2). The first time window T1-A starts at the time of day of t₀ and ends at the time of day of t₁ and has a duration or period of time of t₁−t₀. The second time window T2-A starts at the time of day of t₁ and ends at the time of day of t_{N} and has a duration or period of time of t_{N}-t₁.

FIG. 14B illustrates an example timeline 492 which may be based on an expected care cycle. As shown, the example timeline 492 includes a PI1 and a PI2 which are associated with different time windows. In the particular example, PI1 is associated with a first time window T1-B and third time window T3-B of the timeline 492, and PI2 is associated with a second time window T2-B and a fourth time window T4-B of the timeline 492. For example, the first time window T1-B (including polling interval PI1) starts at 6 am and ends at 10 am and has a duration or period of time of 4 hours. The third time window T3-B (including polling interval PI1) starts at 6 pm and ends at 10 pm and also has a duration or period of time of 4 hours (e.g., 10 pm to 6 pm). The second time window T2-B (including polling interval PI2) starts at 10 am and ends at 6 pm and has a duration or period of time of 8 hours. The fourth time window T4-B (including polling interval PI2) starts at 10 pm and ends at 6 am and has a duration or period of time of 8 hours (e.g., 10 pm to 6 am). In some examples, the polling interval PI1 may be a shorter polling interval than PI2 and may be set based on expected communication times with external circuitry and/or expected care being provided below a threshold. For example, the time windows for polling interval PI1 may be anticipated times when the patient wakes up and/or prior to bed, in which it is predicted (e.g., based on the care usage pattern) that the patient may want to obtain data from the IMD. However examples are not so limited, and expected communication times may be different.

As a specific example, such as in the method 10 of FIG. 1, setting the data event parameter may comprise setting a first polling interval (e.g., PI1) for at least a first time window (e.g., from 6 am to 10 am) and a second time window associated with a sleep-wake status of the patient (e.g., from 6 pm to 10 pm). Setting the data event parameter may further comprise setting a second polling interval (e.g., PI2) for at least a third time window (e.g., 10 pm to 6 am), wherein the second polling interval is greater than the first polling interval and the third time window is associated with care being provided by the IMD based on the care usage pattern. For example, and as shown in association with at least the timeline 492, the first time window may be associated with the onset of an awake state from a sleep state for the patient and the second (e.g., third in the timeline 492) time window may be associated with a time of day prior to sleep onset. The third time window may be associated with at least one of the patient being in a sleep state and a battery parameter of the IMD being above a threshold level.

However, examples are not limited to time windows for polling intervals being set based on a sleep-wake status of the patient. In some examples, setting the data event parameter comprises setting a first polling interval for at least a first time window associated with a time of day after care is provided by the IMD based on the care usage pattern and setting a second polling interval for at least a second time window, the second polling interval being greater than the first polling interval. The second window may be associated with one or more of care being provided by the IMD, the patient being in a sleep state, and a battery parameter of the IMD being above a threshold level. In some examples, the first polling interval may additionally be set for at least one of a third time window and a fourth time window associated with a sleep-wake status of the patient, wherein the third time window is associated with a time of day prior to sleep onset and the fourth time window is associated with onset of an awake state from the sleep state for the patient.

FIG. 14C illustrates an example of revising the polling interval. In the particular example, the timeline 493 illustrates an additional time window associated with PI1 and different durations or periods of time for each of the time windows associated with PI1 and PI2 as illustrated in the timeline 492 of FIG. 14B. The additional time window for PI1 is in the middle of the day, which may, for example, correspond with a lunch break of the patient and in which the patient attempts to access data from the IMD. For example, PI1 is associated with the first time window T1-C, the third time window T3-C, and the fifth time window T5-C. PI2 is associated with the second time window T2-C, the fourth time window T4-C, and the sixth time window T6-C. However, examples are not so limited. In some examples, the additional PI1 time window may be added due to care provided prior to the time window or for other reasons. For example, the adjustment may be in response to the IMD transitioning from expected care cycles to observed care cycles, and/or in response to a current care cycle.

FIG. 14D illustrates another example of revising the polling interval. In the particular example, the timeline 494 includes two time windows for PI1 and PI1, as in the timeline 492 of FIG. 14B, but with adjustments to the start and/or stop time of day and/or to the duration or length of time. Similarly to FIG. 14C, the adjustment may be in response to the IMD transitioning from expected care cycles to observed care cycles, and/or in response to a current care cycle. For example, the IMD may observe the patient may not initiate communication with the IMD prior to going to bed, but does at a lunch break. In response to the observation, the time windows for PI1 and PI2 may be revised. In the example, PI1 is associated with the first time window T1-D and the third time window T3-D. PI2 is associated with the second time window T2-D and the fourth time window T4-D.

Examples are not limited to the number of polling intervals and/or time windows as illustrated by FIGS. 14A-14D. For example, setting the data event parameter may comprise setting a plurality of different polling intervals with respect to different time windows based on the care usage pattern.

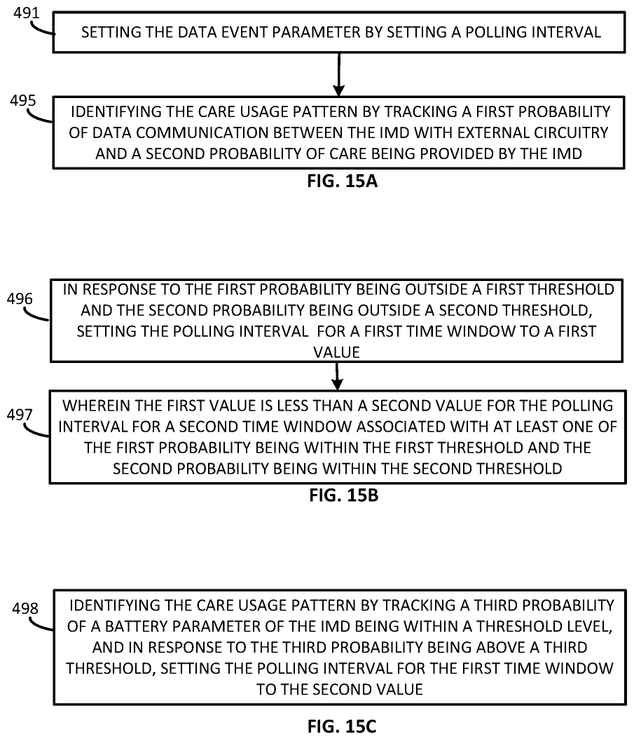

As described above, the care usage pattern may be based on probabilities of communicating with external circuitry at particular times of the day and/or probabilities of care being provided at the particular times of the day. FIGS. 15A-15C provide a non-limiting example of a care usage pattern and tracking probabilities. In some examples, the methods illustrated by FIGS. 15A-15C may comprise part of and/or are example implementations of the method (e.g., 10) illustrated in association with at least FIGS. 1 and 5A-8D and/or the method (e.g., 400) illustrated in association with at least FIGS. 10A-13B.

In some examples, as shown at 491 in FIG. 15A, the method comprises setting a data event parameter by setting a polling interval, and at 495, identifying the care usage pattern comprises tracking a first probability of data communication between the IMD and external circuitry and a second probability of care being provided by the IMD. In some examples, the method of FIG. 15A may further comprise, as shown at 496 in FIG. 15B, in response to the first probability of data communication being outside a first threshold and the second probability of care being outside a second threshold, setting the polling interval for a first time window to a first value. For example, as shown at 497 in FIG. 15B, the first value may be less than a second value for the polling interval for a second time window associated with at least one of the first probability of data communication being within the first threshold and the second probability of care being within the second threshold. Further, in some examples and as shown at 498 in FIG. 15C, the method may comprise identifying the care usage pattern by tracking a third probability of a battery parameter of the IMD being within, e.g., above and/or below, a threshold level, and in response to the third probability being above a third threshold, setting the polling interval for the first time window to the second value.

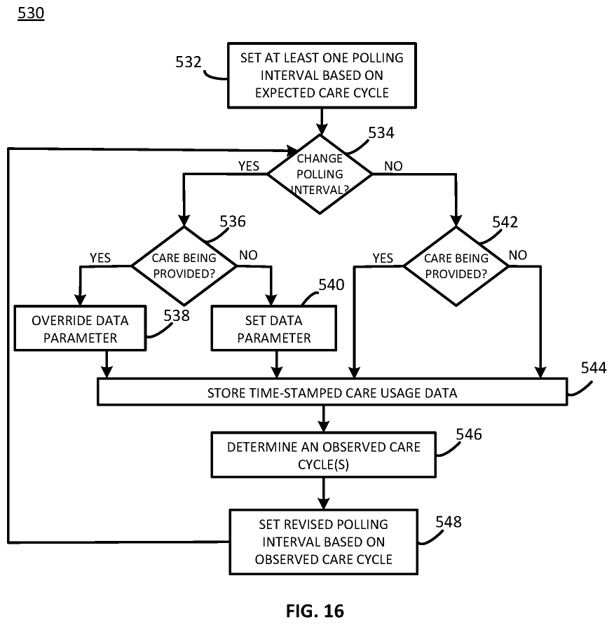

FIG. 16 is a diagram illustrating an example method 530 for setting a configurable polling interval, which may comprise part of and/or is an example implementation of example methods. In some examples, FIG. 16 may comprise part of and/or is an example implementation of the method (e.g., 10) illustrated in association with at least FIGS. 1 and 5A-8D and/or method (e.g., 400) illustrated in association with at least FIGS. 10A-13B. The method 530 may be implemented by an IMD that is programmed with expected care cycles and that transitions, over time, to observed care cycles for setting at least one polling interval for the IMD, such as the IMD 22 of FIG. 2. In some examples, real-time events may override the polling interval set based on expected and/or observed care cycles, and which may, overtime, be used to revise the observed care cycles.

At 532 in FIG. 16, at least one polling interval is set based on at least one expected care cycle of a care usage pattern. In some examples, the care usage pattern may define a plurality of different expected care cycles that indicate different times of the day and/or periods of time (e.g., time windows) that the IMD is likely to provide care to the user and/or that the IMD is likely to communicate with external circuitry. The different care cycles may be selected depending on a set of factors, such as the activity level of the user, the day of the week, the time of the year, among other factors as previously described in connection with FIG. 1. Setting the polling interval may include defining how often the IMD activates the telemetry circuitry and listens for a data communication request from external circuitry. For example, when listening, a data event may be triggered by the IMD in response to receiving the request for the data communication.

Based on the set polling interval, the IMD may have different polling intervals with respect to different time windows of the day. For example, the IMD may have a relaxed (e.g., less often) polling interval during times that external communication is unlikely and/or when care is likely provided by the IMD. At 534 in FIG. 16, the IMD determines if the polling interval is to be changed between different polling intervals. For example, a time window may be reached that is associated with a revised polling interval. The revised polling interval may be set based on the expected care cycle. In response to determining the polling interval is to be changed, at 536, the IMD may determine whether care is being provided by the IMD. In response to a determination that care is being provided, at 538 in FIG. 16, the IMD may override the change to the polling interval and/or override activation of the telemetry circuitry. In response to a determination that care is not being provided, at 540 in FIG. 16, the IMD may change the polling interval and/or activate the telemetry circuitry for triggering a data communication. Referring back to 534 of FIG. 16, in response to determining the polling interval is not to be changed, at 542 in FIG. 16, the IMD may determine whether care is being provided by the IMD. Whether the parameter is changed or not, an indication of whether care is being provided or not, at 544 in FIG. 16, may be stored as time-stamped care usage data by the IMD. The care usage data may be used by the IMD to determine and/or revise observed care cycles, at 546 in FIG. 16. Based on the determined and/or revised care cycles, at 548 in FIG. 16, the IMD may set a revised polling interval.

The method 530 may be repeated over time and used to generate additional and/or revised observed care cycles. In some examples, the care usage engine 27 in FIG. 2 may implement the method 530.

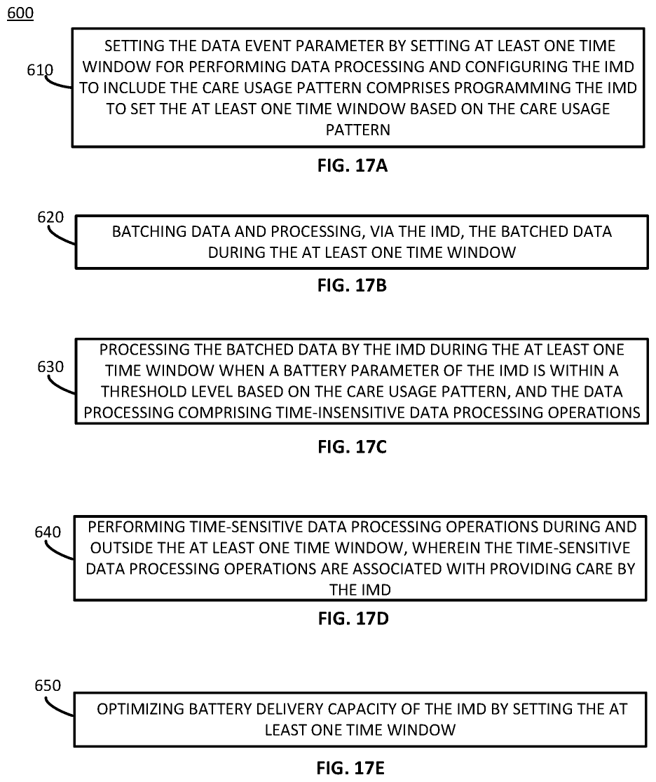

Some examples, alternatively and/or additionally to setting the polling interval, comprise setting a data event parameter by setting a time window for performing data processing. FIGS. 17A-20C are diagrams, which may comprise part of and/or are example implementations of example methods involving setting at least one time window for performing data process. As shown in association with at least FIG. 17A, at 610 the method 600 comprises setting the data event parameter by setting at least one time window for performing data processing by the IMD, and configuring the IMD to include the care usage pattern comprises programming the IMD to set the at least one time window based on the care usage pattern. The method 600 may comprise part of and/or is an example implementation of a method (e.g., 10) as associated with at least FIGS. 1 and 5A-8D in some examples.

In some examples, as shown at 620 in FIG. 17B, the method 600 further comprises batching data and processing, via the IMD, the batched data during the at least one time window. In some examples, the batched data includes data associated with time-insensitive data processing operations. In some examples, time-insensitive data processing operations, as used herein, comprise and/or refer to data operations which may not impact, or impact below a threshold, an ability of the IMD to provide care to the patient. Non-limiting examples of time-insensitive data processing operations include extracting markers or features from previously stored sensor signals and/or other physiological data, training a model (e.g., revised care cycles), computing statistics and/or clinical relevance metric of stored physiological data, marking subsets of physiological data segments based on clinical relevance metrics, selecting a subset of stored physiological data segments based on clinical relevance metric and discarding non-selected segments, defragmenting memory (e.g., moving data elements to increase the size of contiguous unused memory region), executing computer-readable instruction updates, discarding unused or no longer used transient data elements, among other operations.

As shown at 630 in FIG. 17C, in some examples, the method comprises processing the batched data by the IMD during the at least one time window when a battery parameter of the IMD is within a threshold level based on the care usage pattern, and the data processing comprises the time-insensitive data processing operations. As shown at 640 in FIG. 17D, the method 600 may further comprise performing time-sensitive data processing operations during and outside the at least one time window. In some examples, the time-sensitive data processing operations, as used herein, comprise and/or refer to data operations associated with providing care by the IMD.

In some examples, as shown at 650 in FIG. 17E, the method 600 comprises optimizing battery delivery capacity of the IMD by setting the at least one time window. For example, the IMD may use the care usage pattern to predict times of the day that the IMD exhibits the battery parameter within a threshold level, and sets the data event parameter based on the prediction. As previously described, the battery parameter may include a battery capacity (e.g., how much the battery is being utilized), and if the battery capacity is at or below a threshold level, the batched data may be processed. In some examples, the battery parameter may include a battery voltage, and if the battery voltage is at or above a threshold level, the batched data may be processed. Other example battery parameters include battery power delivery capacity, available power, and used power, among others. In some such examples, if the battery power delivery capacity or available power is at or above a threshold level and/or if the used power is at or below the threshold level, the batched data may be processed. The IMD may perform time-sensitive data processing operations based on the data event parameters, which is used to optimize the battery delivery capacity of the IMD.

A battery parameter being within an threshold level, as used herein, may include the battery parameter complying with the threshold level, such as being at or above the threshold level or at or below the threshold level, which indicates the battery is not being fully utilized and/or a data event may be performed. A battery parameter being outside the threshold level, as used herein, may include the battery parameter not complying with the threshold level, such as being below or above the threshold level, which indicates that the battery is being fully utilized and/or the data event should not be performed.

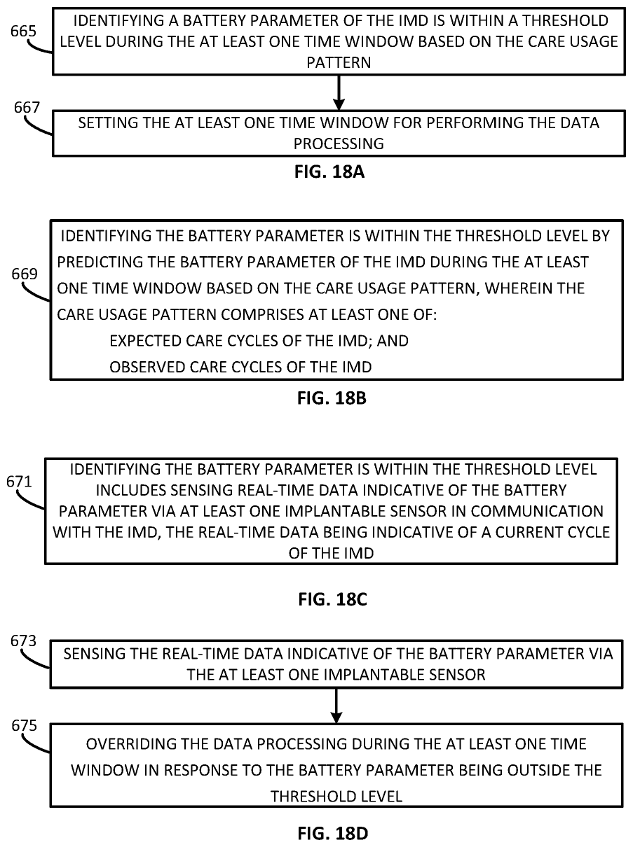

FIGS. 18A-20C may include flow diagrams which may form part of and/or example are implementations of the method (e.g., 600) illustrated in association with at least FIGS. 17A-17E. In some examples, as shown in association with at least FIG. 18A, the method 600 of FIG. 17A further comprises identifying a battery parameter of the IMD is within a threshold level during the at least one time window based on the care usage pattern, at 665, and setting the at least one time window for performing the data processing, at 667. The battery parameter may include battery power delivering capacity and the threshold level may include 2.5V, although examples are not so limited.

In some examples, as shown at 669 in FIG. 18B, the method comprises identifying the battery parameter is within the threshold level by predicting the battery parameter of the IMD during the at least one time window based on the care usage pattern. In some such examples, the care usage pattern comprises at least one of expected care cycles of the IMD and observed care cycles of the IMD.

In some examples, as shown at 671 in FIG. 18C, the method comprises identifying the battery parameter is outside the threshold level by sensing real-time data indicative of the battery parameter via at least one implantable sensor in communication with the IMD, the real-time data being indicative of a current care cycle of the IMD (e.g., real-time event). For example, as shown at 673 in FIG. 18D, the method 600 may further comprise sensing the real-time data indicative of the battery parameter via the at least one implantable sensor, and at 675, overriding the data processing during the at least one time window in response to the battery parameter being outside (e.g., above or below) the threshold level.

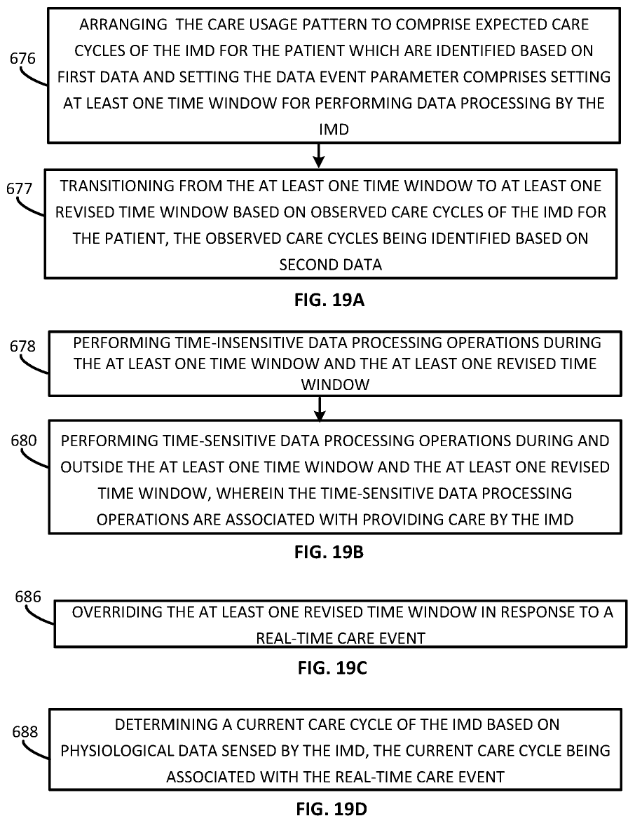

As shown at 676 in FIG. 19A, in some examples, the method may comprise arranging the care usage pattern to comprise expected care cycles of the IMD for the patient which are identified based on first data and setting the data event parameter comprises setting at least one time window for performing data processing by the IMD. In some such examples, as shown at 677 in FIG. 19A, the method 600 of FIG. 17A may further comprise transitioning from the at least one time window to at least one revised time window based on observed care cycles of the IMD for the patient, the observed care cycles being identified based on second data. In some examples, the first data and second data may include at some of substantially the same features and attributes as the first data and second data described in association with at least FIGS. 11A-11C.

In some examples, as shown at 678 in FIG. 19B, the method 600 further comprises, performing time-insensitive data processing operations during the at least one time window and the at least one revised time window, and at 680, performing time-sensitive data processing operations during and outside the at least one time window and the at least one revised time window.

As shown by 686 in FIG. 19C, the method 600 of FIG. 17A may further comprise overriding the at least one revised time window in response to a real-time care event. In some examples, as shown at 688 of FIG. 19D, the method 600 may further comprise determining a current care cycle of the IMD based on physiological data sensed by the IMD, the current care cycle being associated with the real-time care event. For example, overriding the at least one time window may comprise adjusting the time window, such as adjusting the time of day (e.g., start time or stop time) and/or adjusting the length of time.

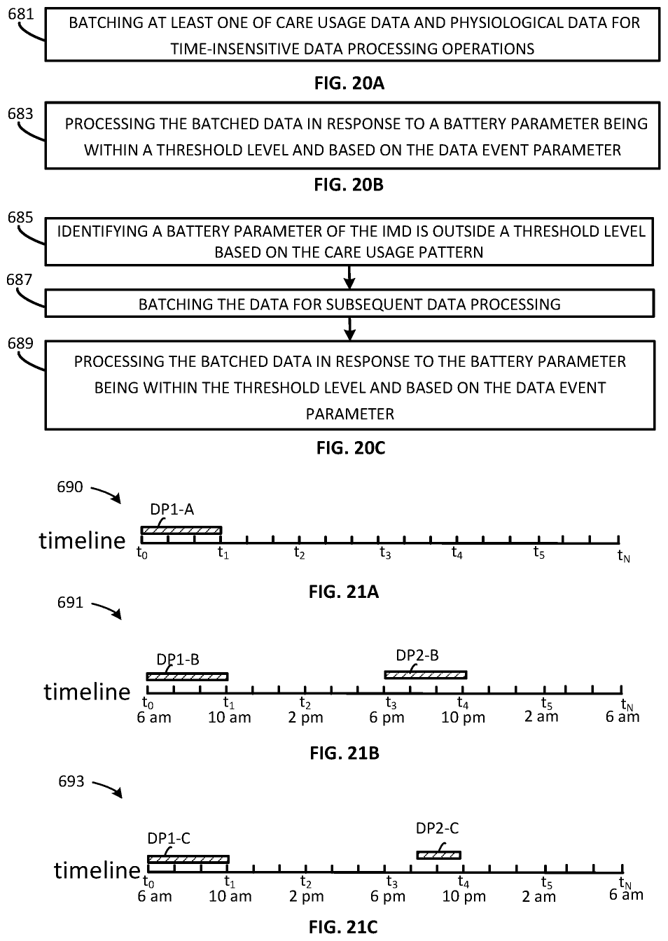

As shown at 681 in FIG. 20A, the method 600 of FIG. 17A may further comprise batching at least one of care usage data and physiological data for time-insensitive data processing operations. For example, the method may comprise processing the batched data in response to a battery parameter being within a threshold level and based on the data event parameter as shown at 683 in FIG. 20B.

In some examples, as shown at 685 in FIG. 20C, the method 600 of FIG. 17A comprises identifying a battery parameter of the IMD is outside a threshold level based on the care usage pattern, and at 687, batching the data for subsequent data processing. At 689, the method 600 further comprises processing the batched data in response to the battery parameter being within the threshold level and based on the data event parameter.

FIGS. 21A-21C are graphs illustrating example configurable time windows for data processing. In some examples, FIGS. 21A-21C may form part of and/or are example implementations of various example methods described herein, including but not limited to the method (e.g., 10) illustrated in association with at least FIGS. 1 and 5A-8D and the method (e.g., 600) illustrated in association with at least FIGS. 17A-17E.

FIG. 21A illustrates a generic timeline 690 showing a first data processing time window (DP1-A). DP1-A starts at the time of day of t₀ and ends at the time of day of t₁, has a duration or period of time of t₁−t₀.

FIG. 21B illustrates an example timeline 691 which may be based on an expected care cycle. As shown the example timeline 691 includes a first data processing time window (DP1-B) and a second data processing time window (DP2-B) at different times of the day. In the particular example, the first data processing time window DP1-B starts at 6 am and ends at 10 am and has a duration or period of time of 4 hours (e.g., 10 am to 6 am). The second data processing time window DP2-B starts at 6 pm and ends at 10 pm and has a duration or period of time of 4 hours (e.g., 10 pm to 6 pm). While the illustrated length of times for data processing are the same, examples are not so limited. In some examples, the different time windows for performing data processing may be set based on a prediction of the IMD exhibiting a battery parameter above and/or below a threshold, which may be based on a prediction of care being provided and processing of time-sensitive data processing operations. The prediction of care being provided may be based on a variety of factor, such as time of day, activity of the user, day of the week, etc., as previously described, as well as disease burden indicators.

FIG. 21C illustrates an example of revising at least one time window for data processing. In the particular example, the timeline 693 illustrates two data processing time windows DP1-C, DP2-C, as in the timeline 691 of FIG. 21B, but with adjustments to the start time and/or stop time of day and/or to the duration or length of time of the second data processing time window DP2-C. However examples are not so limited and the revision may include changes in duration and/or time day for one or more time windows and/or an addition or subtraction of a time window. For example, the additional time window may be added due to care provided or for other reasons. Accordingly, the adjustment may be in response to the IMD transitioning from expected care cycles to observed care cycles, and/or in response to a current care cycle.

Examples are not limited to the number of time windows and/or time windows as illustrated by FIGS. 21A-21C. For example, setting the data event parameter may comprise setting a plurality of different time windows based on the care usage pattern.

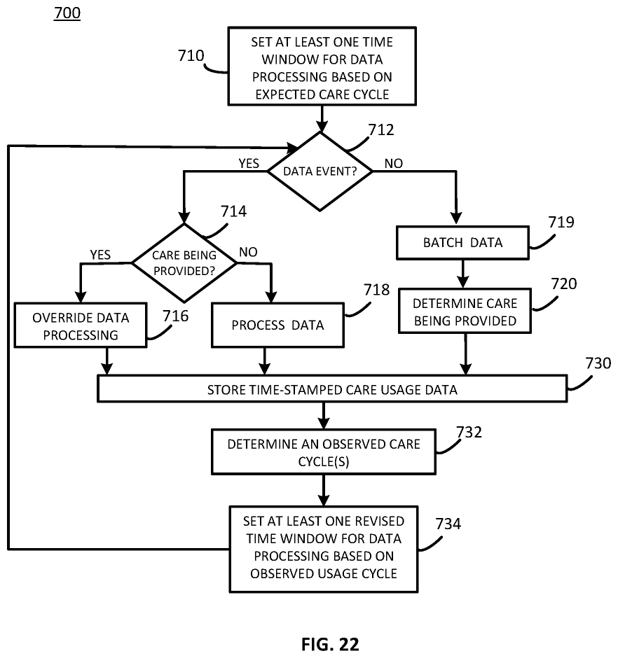

FIG. 22 is a diagram illustrating an example method 700 for setting a configurable time window for performing data processing, which may comprise part of and/or is an example implementation of example methods. In some examples, FIG. 22 illustrates an example implementation of the method (e.g. 10) illustrated in association with at least FIGS. 1 and 5A-8D and/or method (e.g., 600) illustrated in association with at least FIGS. 17A-17E. The method 700 may be implemented by an IMD that is programmed with expected care cycles and that transitions, over time, to observed care cycles for setting at least time window for performing data processing by the IMD, such as the IMD 22 illustrated by FIG. 2. In some examples, real-time events may override the at least one time window set based on expected and/or observed care cycles, and which may, overtime, be used to revise the observed care cycles.

At 710 in FIG. 22, the example method comprises setting at least one time window for performing data processing based on at least one expected care cycle of a care usage pattern. In some examples, the care usage pattern may define a plurality of different expected care cycles that indicate different times of the day and/or period of time (e.g., time windows) that the IMD is likely to provide care to the user and/or that the IMD is likely to exhibit a battery parameter outside a threshold level. The different care cycles may be different depending on the activity level of the user, the day of the week, the time of the year, among other factors, such as the set of factors previously described by FIG. 1. In some examples, setting the at least one time window may define when the IMD performs time-insensitive data processing operations.

At 712 in FIG. 22, the IMD determines if the data event is to occur based on the at least one time window and a time of day. For example, in response to reaching a time window associated with data processing, the IMD may perform the data processing operation. In response to determining the data event is not set to occur, at 719 in FIG. 22, the IMD may batch data for subsequent data processing. In some examples, the data being batched may be associated with time-insensitive data operations and the IMD may process data (which may be overlapping with the batch data) for time-sensitive data operations at any time, such as operations used to determine whether to and/or otherwise associated with providing care to the patient. At 720 in FIG. 22, the IMD may determine whether or not care is being provided by the IMD. Referring back to 712, in response to determining the data event is to occur, at 714 in FIG. 22, the IMD may determine whether care is being provided by the IMD. In response to a determination that care is being provided (or otherwise determining the IMD exhibits a battery parameter outside a threshold), at 716 in FIG. 22, the IMD may override the data processing event and batch the data for subsequent data processing. In response to a determination that care is not being provided, at 718 in FIG. 22, the IMD may perform the time-insensitive data processing operation. An indication of whether care is being provided or not, at 730 in FIG. 22, may be stored as time-stamped care usage data. The care usage data may be used by the IMD to determine and/or revise observed care cycles, at 732 in FIG. 22. Based on the observed care cycles, at 734 in FIG. 22, the IMD may set at least one revised time window for data processing.

The method 700 may be repeated over time and used to generate additional and/or revised observed care cycles. In some examples, the care usage engine 27 of FIG. 2 may implement method 700.

Some example methods, systems and/or devices may involve programming an IMD (e.g., IMD 22 in FIG. 2) to identify or select the care usage pattern and set a data event parameter using data, such as data from at least one implantable sensor which may form part of or be associated with the IMD. In some examples, such programming may comprise determining which internally sensed data is correlated with, and/or acts as a surrogate for, information typically used to identify the care usage pattern and/or set the data event parameter, such as the above identified patterns of data sensed by the at least one implantable sensor. In at least some examples, the programming may include or involve a data model. In some examples, external circuitry may determine the above identified patterns and program the IMD using the identified patterns, such as by constructing a data model and programming the data model. In other examples, the IMD determines the identified patterns and/or determines the patterns in combination with external circuitry.

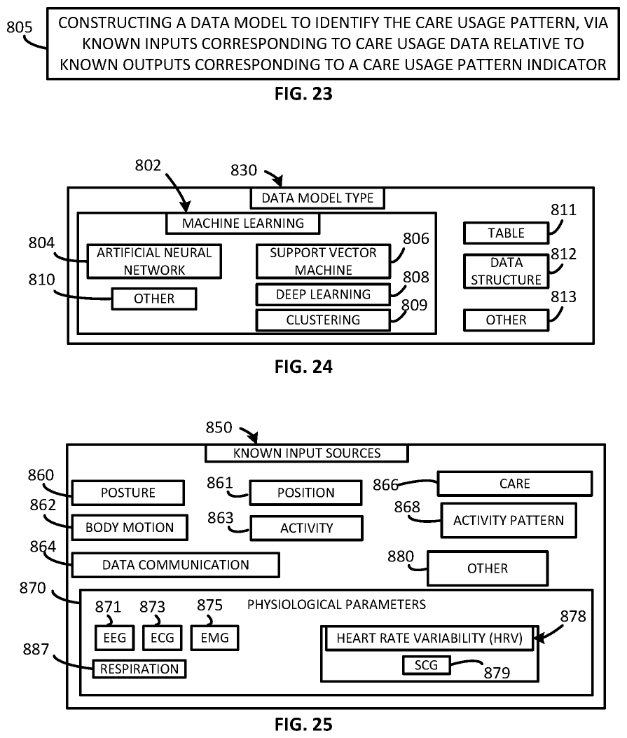

With this in mind, the following example implementations in FIGS. 23-29 provide a framework of parameters, inputs, input sources, outputs, signals, devices, methods, etc., as part of providing an IMD to set configurable data event parameter(s). Some of the example implementations comprise a data model or parameters, inputs, etc. associated with use of a data model, while some example implementations omit use of a data model. Regardless of whether a particular example includes a data model or not, it will be understood that the various parameters, inputs, input sources, signals, devices, methods may be combined in various permutations to achieve a desired array of inputs, outputs, etc. by which the IMD may be programmed or otherwise constructed to identify an expected care cycle, observed care cycle, and/or current care cycle via internally sensed data.

FIG. 23 is a diagram, which may comprise part of and/or is an example implementation of example methods (e.g., method 10 of FIG. 1). As shown at 805 in FIG. 23, the method may include constructing a data model to identify the care usage pattern based on known inputs corresponding to at least one of a care usage time and an amount of care provided to the patient by the IMD relative to known outputs corresponding to a care usage pattern indicator. In some examples, the known inputs may include or be associated with the set of factors, as previously described by FIG. 1. In some such examples, the data model may be constructed via training the data model.

In some examples, the data model may comprise at least one of the data model types 830 shown in FIG. 24. Accordingly, as shown in FIG. 24, in some examples the data model types 830 may comprise a machine learning model 802, which may comprise an artificial neural network 804, support vector machine (SVM) 806, deep learning 808, cluster 809, and/or other models 810. However, examples are not limited to machine learning models 802 and may include a correlation table 811, a data structure 812, among other models 813, and which may include the above described patterns and/or a probabilistic approach, which may be known inputs.

In some examples, the artificial neural network 804 may estimate a function(s) that depend on inputs. In some such examples, one or more layers of artificial neurons may receive input data and generate output data. The inputs and outputs may comprise the data sensed by the at least one implantable sensor and/or functions related to such data or other functions. Neural networks may comprise networks such as, but not limited to, learning networks (e.g., deep, deep structured, hierarchical, and the like), convolutional, auto-type networks (e.g., auto-encoder, auto-associator), Diablo networks, and neural network models (e.g., feedforward, recurrent).

In some examples, the SVM 806 may utilize a linear classification. This classification may act to separate the data points into classes based on distance of the data points from a hyperplane. In some examples, the hyperplane is arranged to maximize the distances from the hyperplane to the nearest data points on either side of the hyperplane. This arrangement may group points located on opposite sides of the hyperplane into different classes. However, in some examples, the SVM 806 may comprise a nonlinear classification that separates the data points with a hyperplane in a transformed feature space. The transformed feature space may be determined by one or more kernel functions, including nonlinear kernel functions. In some examples, the SVM 806 is a multiclass SVM that separates data points into more than two classes, which may reduce a multiclass problem into multiple binary classification problems.

In some examples, the deep learning 808 may comprise models such as, but not limited to, convolutional networks (e.g., deep belief, neural), belief networks, Boltzmann machines, deep coding networks, stacked auto-encoders, stacking networks (e.g., deep or tensor deep), hierarchical-deep models, deep kernel machines, and the like. It will be understood that such examples may comprise variants and/or combinations of the above-noted example networks.

In some examples, per type 809, the data model may comprise a clustering method(s), which may comprise hierarchical clustering, k-means clustering, density-based clustering, and the like. In some examples, the hierarchical clustering may be used to construct a hierarchy of clusters of sensed data. In some such examples, the hierarchical clustering utilizes a “bottom up” approach (e.g., agglomerative) wherein each data point starts in its own cluster, and pairs of clusters are merged at progressively higher levels of the hierarchy. However, in some examples, the hierarchical clustering utilizes a top-down approach in which all data points start in one cluster, and then clusters are split at progressively lower levels of the hierarchy.

In some examples, the k-means clustering implementation may comprise placing the sensed data into k clusters, where k is an integer equal or greater than two. Via such clustering, each data point belongs to a cluster having a mean that is closer to the data point than any means of the other clusters. However, in some examples, a machine learning model (MLM) may comprise density-based clustering, which may be used to group together data points that are close to one another, while identifying as outliers any data points that are far away from other data points.

In some examples, as represented per “other” type 810 in FIG. 24, a MLM may comprise a mean-shift analysis that may be used to determine the maxima of a density function based on discrete data sampled from that function.

In some examples, as represented per “other” type 810 in FIG. 24, a MLM may comprise structured prediction techniques and/or structured learning techniques. Such techniques may be used to predict structured objects and/or structured data, such as structured patient-volitional data and patient non-volitional data. In some such examples, such structured prediction and/or structured learning techniques may comprise graphical models, probabilistic graphical models, sequence labeling, conditional random fields, parsing, collective classification, bipartite matching, Bayesian networks or models, and the like. It will be understood that such examples comprise variants and/or combinations of the above-noted example techniques.

In some examples, a MLM may comprise anomaly detection and/or outlier detection that may be used to identify data that does not conform to an expected pattern or are otherwise distinct from other data in a dataset.

In some examples, machine learning model may comprise learning methods that incorporate a plurality of the machine learning methods.

It will be understood that at least some example methods (and/or devices) of the present disclosure may sense physiological data, and set the data event parameter without use of a constructed data model and/or trained data model, such as but not limited to, a machine learning model. Further, the data model may be constructed on a per-patient basis and/or a representative patient basis.

In some examples a method may comprise implementing construction of a data model at least partially via at least one external resource, in communication with the IMD, according to at least some external data. In some such examples, the external data comprises data indicative of data communication between the IMD and external circuitry (such as detected by external telemetry circuitry) and/or data indicative of care being provided to the user. The external data may be time-stamped by the external resource or by the IMD.

FIG. 25 is a block diagram schematically representing at least some example known input sources 850. The input sources 850 may comprise external sources and/or internal sources, such as data sensed by the at least one implantable sensor of a particular IMD or a plurality of IMDs. In some example methods, a data model may be constructed via providing known inputs to the data model based on known input sources 850. The known input sources 850 may comprise signals indicative of posture 860, position 861, activity 863, motion 862, data communications 864, care being provided 866, activity patterns 868, physiological parameters 870, and other inputs 880 including time of day, time zone, time of year, day of the week, etc. In some examples, the data indicative of care being provided 866 may comprise a period of time of providing care, a type of care provided, and/or an amount or amplitude of care provided. In some examples the data indicative of data communications 864 may include periods of time and/or times of the day of data communication between the external circuitry and the IMD or representative external circuitries and representative IMDs. In some examples, the activity patterns 868 may comprise observed activity patterns of the patient and/or observed patterns of representative patents. In some examples, observed activity patterns of the patient may be identified or determine using internally sensed data, such as data sensed by an implantable sensor as described by FIGS. 4A-4B and/or using a care usage engine 27 as described by FIGS. 9A-9C.

In some examples, the known input sources 850 may comprise data indicative of expected or known patterns of sensed data, such as patterns of motion and posture, as well as care provide, and associations between the same, as described above.

In some examples, the physiological parameters 870 may comprise a respiration signal 887, a respiration rate variability signal, a heart rate variability signal 878, in which may be obtained from seismocardiography sensing (SCG) 879, an EEG parameter 871, ECG parameter 873, and/or an EMG parameter 875. Other inputs sources 850 may comprise ballistocardiography sensing (BCG), and/or accelerocardiograph sensing (ACG). In some examples, the SCG, BCG, ACG sensing may be provided via an implanted acceleration sensor or via other types of implantable sensors. In some examples, the physiological parameters 870 may be indicative of care being or about to be provided by the IMD.

In some examples, the motion 862 may be used to obtain at least one of the physiological parameters 870. For example, motion data sensed by an acceleration sensor may be used to determine respiratory information, as further described herein. In some examples, the respiration information is determined by sensing, via the acceleration sensor, rotational movement associated with a respiratory body portion of the patient with the IMD implanted, with the rotational movement being caused by breathing. In some such examples, the respiratory body portion may comprise a chest wall and/or abdominal wall of the patient, and the motion may include chest motion, such as chest wall motion comprising a rotational movement of the chest wall and/or rotational movement of an abdominal wall or portion of the abdomen indicative to respiratory information, and as described within U.S. patent application Ser. No. 16/977,664, filed on Sep. 2, 2020, and entitled “RESPIRATION DETECTION”, the entire teachings of which is incorporated herein by reference in its entirety.

The known input sources 850 may include various external and internal data sources, such as the implantable sensor of the IMD, implantable sensors of other IMDs, external databases which store data from a plurality of IMDs, such as various data for the respective IMD or for a plurality of IMDs. Accordingly, the data model may be constructed for the particular patient (e.g., per-patient basis) or representative number of patients (e.g., representative patient basis). Additionally, the data model may be updated overtime using feedback data from the particular IMD and/or a plurality of IMDs.

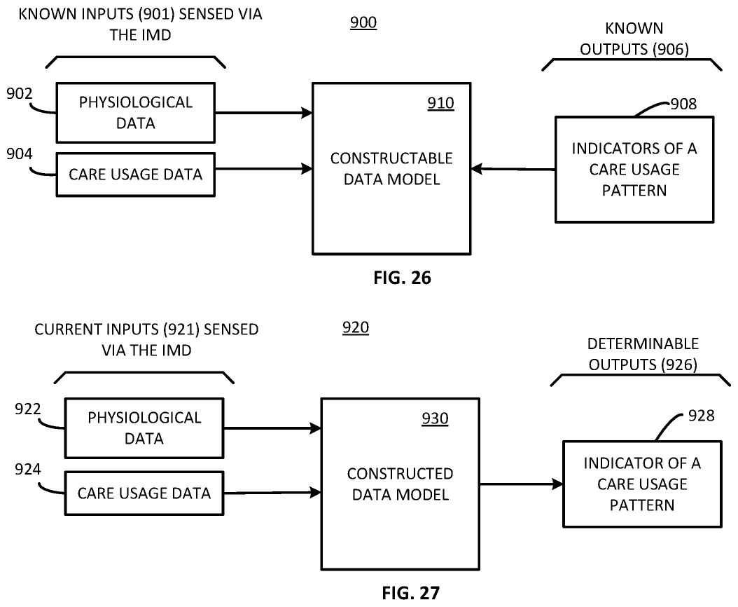

FIG. 26 is a diagram schematically representing an example method 900 of constructing a data model for use in later identifying a care cycle for setting a data event parameter. As shown in FIG. 26, the method 900 comprises constructing a data model by providing known inputs 901 and known outputs 906 to the constructable data model 910. The known inputs 901 may be obtained and/or sensed via the IMD, such as by at least one implanted sensor of a particular IMD, and/or via implanted sensors of a plurality of representative IMDs. In some examples, the known outputs 906 may be obtained and/or sensed via at least one sensor located external to the patient's body, herein sometimes referred to as “an external sensor”. However, examples are not so limited and the known outputs 906 may be based on data internal to the IMD.

The known inputs 901 may comprise physiological data 902 and care usage data 904. Example physiological data 902 may comprise motion and posture data sensed using an acceleration sensor and care usage data 904 may comprise data indicative of a type, an amount, and a duration of care provided. In some examples, the physiological data 902 and/or and care usage data 904 may be time-stamped and/or time data (e.g., time of day, duration of time, etc.) may additional provided as known inputs.

The known outputs 906 may comprise indicators of a care usage pattern 908. For example, the indicators of the care usage pattern 908 may comprise times of the day and durations of care being provided by the IMD, a battery parameter being within and/or outside a threshold level, and/or data communications associated with and/or between the IMD and external circuitry, which indicate when to set the data event parameter. In some examples, the known outputs 906 (e.g., the indicators 908) may comprise data measured externally from the IMD, such as by the at least one external sensor. The at least one external sensor may comprise external telemetry circuitry configured to sense data communications by the IMD and/or an external sensor used to detect an indicator of a disease burden, such as heart rate sensor, pulse oximeter, blood oxygen desaturation, airflow sensor, among other types of sensors. However, examples are not so limited. For example, with observed care cycles, the known outputs 906 may be obtained by the IMD.

As previously described, constructing the data model may comprise training a data model, such as one of the data models in data model types 830 in FIG. 25 with one of the example data model types comprising a machine learning model 802. By providing such known inputs 901 and known outputs 906 to the constructable data model 910, a constructed data model 930 (FIG. 27) may be obtained. As noted elsewhere, the constructable data model 910 (FIG. 26) may comprise a trainable MLM and the constructed data model 930 (FIG. 27) may comprise a trained MLM. In the particular example, the constructable data model 910 (FIG. 26) is trained (forming the constructed data model 930) using data from the particular IMD, and may be said to be “per-patient”. However examples are not so limited, and may include constructing a data model that is “representative patient-based”. Once constructed, the data model 930 as illustrated by FIG. 27, may be used in a method 920 in which currently sensed inputs 921 are fed into the constructed data model 930, which produces an output 926 as an indicator 928 of a care usage pattern. The indicator 928 may be used to set a data event parameter. In some examples, the constructed data model 930 uses the inputs 921 as at least a portion of the set of factors, as described at least by FIG. 1, to select a care cycle from the constructed data model 930 and based on the selected care cycle, to set the data event parameter. The care cycle may be selected by pattern matching the set of factors to the particular care cycle.

FIG. 27 is a diagram schematically representing an example method 920 of using a constructed data model 930 for setting a data event parameter using internal measurements, such as via an implanted sensor. As shown in FIG. 27, currently sensed inputs 921 are fed into the constructed data model 930 (e.g., trained MLM), which then produces determinable outputs 926, such as the indicator 928 of the a care usage pattern, which is based on the current inputs 921. In some examples, the current inputs 921 include physiological data 922 and care usage data 924 obtained via the IMD and the current inputs 921 correspond to the types of known inputs 901 obtained via the IMD.

In some examples, just one or some of the inputs 921 may be used, while all of the inputs 921 may be used in some examples.

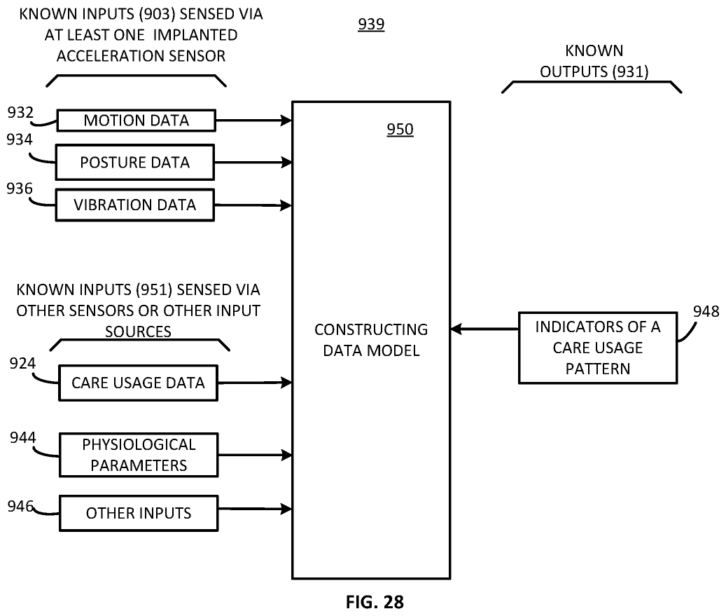

FIG. 28 is diagram schematically representing an example method 939 of constructing a data model. Method 939 may comprise at least some of substantially the same features and attributes as method 900 in FIG. 26. For example, the known inputs 901 of FIG. 26 may comprise the known inputs 903 and 951 of FIG. 28, which include inputs sensed by at least one implantable acceleration sensor and other inputs sensed or otherwise provided by other sensors or input sources. The known outputs 931 may include those previously described in connection with FIG. 26, e.g., the indicators of a care usage pattern 948, which may be indicative of times when care is not predicted to be provided, a battery parameter is predicted to be above and/or below a threshold level, and/or data communications are predicted to occur between IMD and externa circuitry. In some examples, the known inputs 903, 951 may be sensed using internal sensors to the IMD. In some examples, the known inputs 903, 951 may further or alternatively include data sensed by external data sources, such as sensors of other IMDs and/or patterns of known inputs that indicate the care being provided, a battery level, and/or data communications to occur by the IMD. In some examples, using both the internally measured known inputs and the externally measured known inputs may enhance accuracy, robustness, etc., in constructing the data model 950.

As shown in association with at least FIG. 28, the known inputs 903 sensed via the at least one implantable sensor (e.g., an acceleration sensor) comprise motion data 932, posture data 934, and vibration data 936, which may include other data such as position data and activity data. In some examples, the motion data 932, posture data 934, and vibration data 936 may comprise the physiological data 902 in FIG. 26. The known inputs 951 sensed via the other sensor circuitry may comprise care usage data 924, such as time, amplitude, and duration of care being provided, and physiological data 944. Additionally, other inputs 946 may be provided to construct the data model, such as a temporal parameter.

In some examples relating to at least FIG. 28, just one or some of the inputs 903 and just some of the inputs 951 may be used, while all of the inputs 903 and/or all of the inputs 951 may be used in some examples. For example, the other inputs 946 may include data from external circuitry indicative of at least some of the set of factors previously described by FIG. 1, such as sleep pattern, activity patterns, dietary intake, weather, time of the year, day of the week, etc. However, examples are not so limited. For example, the IMD may determine the sleep pattern and/or activity pattern using internally sensed data from the at least one implantable sensor, such the acceleration sensor.

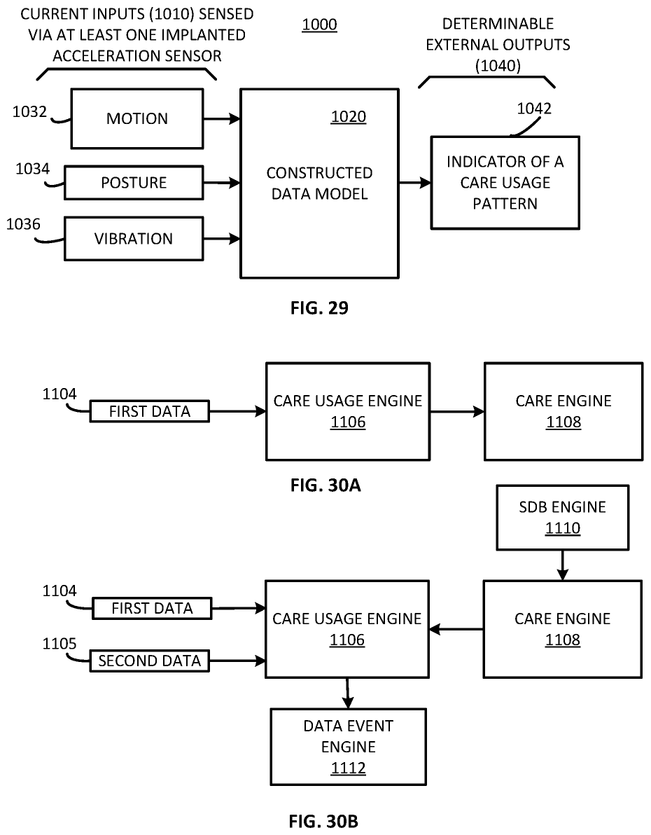

FIG. 29 is a diagram schematically representing an example method 1000 of using a constructed data model 1020. The constructed data model 1020 is obtained via the method 939 in FIG. 28 via constructing data model 950, which includes the additional known inputs 951. As shown in FIG. 29, currently sensed inputs 1010 are fed into the constructed data model 1020 (e.g., a trained MLM), which then produces a determinable output 1040, such as an indicator 1042 of the care usage pattern, which is based on the current inputs 1010. In some examples, the current inputs 1010 are obtained via the at least one implanted sensor (e.g., acceleration sensor), which include motion data 1032, posture data 1034, and vibration data 1036 from the acceleration sensor (or other input sources) and the current inputs 1010 correspond to the types of known inputs 903 obtained via the at least one implantable sensor. Although not illustrated, in some examples, the current inputs 1010 may additionally comprise at least one input sensed via other sensors or sources, such as those similar to the known inputs 951 in FIG. 29.

FIGS. 30A-30B are block diagrams schematically representing example IMD systems including a care usage engine 1106. The care usage engine 1106 illustrated by FIGS. 30A-30B may be an implementation of the care usage engine 27 that forms an IMD system 20 with an IMD 22 and at least one implantable sensor 25, as illustrated by FIG. 2. Accordingly, the care usage engine 1106 may select a care cycle of a care usage pattern using first data 1104 and optionally second data 1105, as previously described and illustrated in FIG. 30B, and set at least one data event parameter based on the care cycle.

In some examples, the care usage engine 1106 may be programmed to set or revise the data event parameter based on communication with another engine that controls an operational feature, such as based on care provided by the IMD. For example, as shown in association with at least FIG. 30A, the IMD system includes care engine 1108 that communicates with the care usage engine 1106 to revise a set data event parameter, which may be based on or in response to real-time events. The care engine 1108 may provide care to the patient. Providing care may include, but is not limited to, measuring and/or or monitoring physiological data, providing information (e.g., feedback, suggestions, alerts) to the patient or a caregiver based on the physiological data, and/or delivering therapy to the patient, and various combinations thereof. In some examples, delivering therapy by the IMD comprises application of a stimulation signal to the patient and/or delivery of fluid to the patient, however examples are not so limited. The care engine 1108 may communicate data to the care usage engine 1106 indicative of currently providing (or not) care to the patient, and the care usage engine 1106 may use the data to generate or revise observed care cycles and/or to override a data event and/or data event parameter.

In some examples, and as further illustrated by FIG. 30B, the IMD system may further comprise an SDB engine 1110 which may include a sleep detection feature to identify SDB. Non-limiting examples of some features implemented by the SDB engine 1110 in accordance with systems and methods of the present disclosure may comprise at least some of substantially the same features and attributes as described within at least: U.S. Patent Publication No. 2019/0160282, published on May 30, 2019, and entitled “ACCELEROMETER-BASED SENSING FOR SLEEP DISORDED BREATHING (SDB) CARE”; U.S. patent application Ser. No. 16/978,470, filed Sep. 4, 2020, and entitled “SLEEP DETECTION FOR SLEEP DISORDERED BREATHING (SDB) CARE”; and/or U.S. patent application Ser. No. 16/978,283, filed Sep. 4, 2020, and entitled “SYSTEMS AND METHODS FOR OPERATING AN IMPLANTABLE MEDICAL DEVICE BASED UPON SENSED PHYSICAL ACTION”, the entire teachings of which are incorporated herein by reference in their entireties.

In some examples, the SDB engine 1110 may detect a disease burden indicator, such as detecting an indicator of sleep apnea. Non-limiting examples of some features implemented by the SDB engine 1110 in accordance with systems and methods of the present disclosure may comprise at least some of substantially the same features and attributes for detecting a disease burden indicator as described within at least: U.S. Provisional Patent Application No. 63/056,241, filed on Jul. 24, 2020, and entitled “DISEASE BURDEN INDICATION”; and U.S. Provisional Patent Application No. 63/089,118, filed on Oct. 8, 2020, and entitled “IDENTIFYING A PRESENCE-ABSENCE STATE OF A MAGNETIC RESONANCE IMAGING SYSTEM”, the entire teaching each of which are incorporated herein by reference in their entireties.

In some examples, the IMD may comprise an SDB care device having an IPG. In some such examples, care provided by the care engine 1108 may comprise delivering stimulation therapy (e.g., delivering a stimulation signal) when the patient is in a sleep state. In some examples, the care usage engine 1106 may identify the care usage pattern based on the sleep-wake status of the patient. For example, the care usage pattern may include and/or be based on an expected sleep-wake status pattern, an observed sleep-wake status based on a historically observed sleep-wake status pattern, and a current sleep-wake status. As an example, a first polling interval may be set for at least a first time window associated with an awake state of the patient (e.g., a time of the day prior to sleep onset or onset of an awake state from a sleep state). A second polling internal may be set for at least a second time window associated with a sleep state of the patient, where the second polling interval is greater than the first polling interval.

In some examples that include an SDB care device, the observed sleep-wake status and/or the current sleep-wake status by may be determined by sensing physiological data via at least one implantable sensor in communication with the IMD, such as the physiological data described above.

Examples are not limited to SDB care devices and may comprise other neurostimulators, sensing, and/or cardiac care devices. Other example sensing and/or stimulating devices may be directed to sensing and/or simulating for urinary and/or pelvic disorders.

In some examples, for a neurostimulator, and the care provided comprises delivering neurostimulation by the neurostimulator. In some such examples, the care usage pattern may be identified based on an intensity of neurostimulation delivered, a time of day of delivery of the neurostimulation, and/or a time of day of data communication between the IMD and external circuitry.

As an example, with a neurostimulator, a first polling interval may be set for at least a first time window associated with the time of day of delivery of the neurostimulation based on the care usage pattern, the first time window following the time of day of delivery. A second polling interval may be set for at least a second time window, the second polling interval being greater than the first polling interval. In some examples, the care usage pattern may additionally be identified based on the sleep-wake status of the patient. For example, the first polling interval may be set for the first time window and at least a third time interval, where the third time interval is associated with a time of day prior to sleep onset for the patient and/or an onset of an awake state from a sleep state for the patient.

For a pacer or other cardiac care device, the IMD may provide care by delivering cardiac stimulation therapy to the patient. In some such examples, the care usage pattern may be identified based on at least one of an intensity of cardiac stimulation delivered, a time of day of delivery of the cardiac stimulation, and a time of day of data communication between the IMD and external circuitry.

As an example, with the cardiac care device, a first polling interval may be set for at least a first time window associated with the time of day of delivery of the cardiac stimulation based on the care usage pattern, the first time window following the time of day of delivery. A second polling interval may be set for at least a second time window, the second polling interval being greater than the first polling interval. In some examples, the care usage pattern may additionally be identified based on the sleep-wake status of the patient. For example, the first polling interval may be set for the first time window and at least a third time interval, where the third time interval is associated with a time of day prior to sleep onset for the patient and/or an onset of an awake state from a sleep state for the patient.

In some examples the IMD comprises an implanted infusion pump that provides care by delivering fluid (e.g., medicine) to the patient. In some such examples, the care usage pattern may be identified based on at least one of an amount of fluid delivered, a time of day of delivery of the fluid, and a time of day of data communication between the IMD and external circuitry.

As an example, with the implanted infusion pump, a first polling interval may be set for at least a first time window associated with the time of day of delivery of the fluid based on the care usage pattern, the first time window following the time of day of delivery. A second polling interval may be set for at least a second time window, the second polling interval being greater than the first polling interval. In some examples, the care usage pattern may additionally be identified based on the sleep-wake status of the patient. For example, the first polling interval may be set for the first time window and at least a third time interval, where the third time interval is associated with a time of day prior to sleep onset for the patient and/or an onset of an awake state from a sleep state for the patient.

In related and non-limiting examples, a data event may be performed by the IMD based on the data event parameter. For example, data may be communicated to external circuitry, such as external device 26 illustrated by FIG. 2, and/or data processing operations may be performed. Referring back to FIGS. 30A-30B, the care usage engine 1106 and/or other engine of the IMD, such as the illustrated data event engine 1112, may be programmed to perform the data event. As a point of reference and referring back to FIG. 2, the IMD 22 may be configured to interface (e.g., via telemetry) with a variety of external devices. For example, the external device 26 may include, but is not limited to, a patient remote, a physician remote, a clinician portal, a handheld device, a mobile phone, a smart phone, a desktop computer, a laptop computer, a tablet personal computer, etc. The various data captured by the IMD 22 may be used as part of a software application, uploaded to a database or other external storage source (e.g., the cloud, a website), etc. The external device 26 may include a smartphone or other type of handheld (or wearable) device that is retained and operated by the patient to whom the IMD 22 is implanted. In some examples, the external device 26 may include a personal computer or the like that is operated by a medical caregiver for the patient. The external device 26 may include a computing device designed to remain at the home of the patient or at the office of the caregiver.

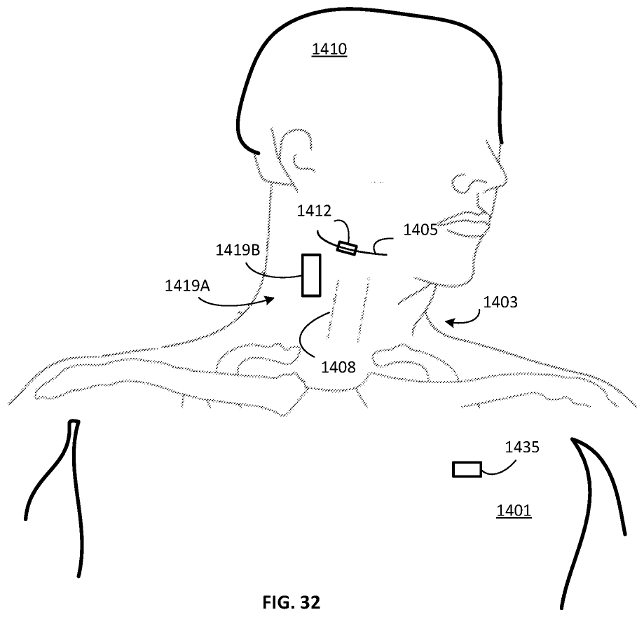

FIG. 31 is a diagram including a front view of an example device 1411 (and/or example method) implanted within a patient's body 1410. In some examples, the device 1411 may comprise an IMD such as (but not limited to) an implantable pulse generator (IPG) 1433 with IMD including a sensor 1435. In some examples, the device 1411 comprises at least some of substantially the same features and attributes as IMD 22 (including the at least one implantable sensor 25), as previously described in association with at least FIG. 2). Accordingly, in some examples, sensor 1435 may comprise at least an acceleration sensor (e.g., 110 in FIG. 4A) having at least some of substantially the same features and attributes as previously described in association with at least FIGS. 1-30B. For example, FIG. 31 illustrates an example IMD by which FIGS. 1-30B and/or FIGS. 32A-35 may be implemented.

As further shown in FIG. 31, the device 1411 comprises a lead 1417 including a lead body 1418 for chronic implantation (e.g., subcutaneously via tunneling or other techniques) and to extend to a position adjacent a nerve (e.g., hypoglossal nerve 1405 and/or phrenic nerve 1406). The lead 1417 may comprise a stimulation electrode 1412 to engage the nerve (e.g., 1405, 1406) in a head-and-neck region 1403 for stimulating the nerve to treat a physiologic condition, such as sleep disordered breathing like obstructive sleep apnea, central sleep apnea, multiple-type sleep apneas, etc. The device 1411 may comprise circuitry, power element, etc. to support control and operation of both the sensor 1435 and the stimulation electrode 1412 (via lead 1417). In some examples, such control, operation, etc. may be implemented, at least in part, via a control portion (and related functions, portions, elements, engines, parameters, etc.) such as described later in association with at least FIGS. 32A-35.

With regard to the some examples of the present disclosure, in some examples, delivering stimulation to an upper airway patency nerve (e.g., a hypoglossal nerve 1405) via the stimulation electrode 1412 is to cause contraction of upper airway patency-related muscles, which may cause or maintain opening of the upper airway (1408) to prevent and/or treat obstructive sleep apnea. Similarly, in some examples such electrical stimulation may be applied to a phrenic nerve 1406 via the stimulation electrode 1412 to cause contraction of the diaphragm as part of preventing or treating at least central sleep apnea. It will be further understood that some example methods may comprise treating both obstructive sleep apnea and central sleep apnea, such as but not limited to, instances of multiple-type sleep apnea in which both types of sleep apnea may be present at least some of the time. In some such instances, separate stimulation leads 1417 may be provided or a single stimulation lead 1417 may be provided but with a bifurcated distal portion with each separate distal portion extending to a respective one of the hypoglossal nerve 1405 and the phrenic nerve 1406.

In some examples, the device 1411 may treat multiple-type sleep apnea using at least some of substantially the same features and attributes as described within U.S. Patent Publication No. 2020/0147376, published on May 14, 2020, and entitled “MULTIPLE TYPE SLEEP APNEA”, the entire teachings of which is incorporated herein by reference in its entirety. In some examples, the device 1411 may treat and/or stimulate the ansa cervicalis (AC)-related nerve to maintain and/or restore upper airway patency using at least some of substantially the same features and attributes as described within U.S. Provisional Patent Application No. 63/029,446, filed on May 23, 2020, and entitled “SINGLE OR MULTIPLE NERVE STIMULATION TO TREAT SLEEP DISORDERED BREATHING”, the entire teachings of which is incorporated herein by reference in its entirety.

In some such examples, the contraction of the hypoglossal nerve and/or contraction of the phrenic nerve caused by electrical stimulation comprises a suprathreshold stimulation, which is in contrast to a subthreshold stimulation (e.g., mere tone) of such muscles. In one aspect, a suprathreshold intensity level corresponds to a stimulation energy greater than the nerve excitation threshold, such that the suprathreshold stimulation may provide for higher degrees (e.g., maximum, other) of upper-airway clearance (i.e., patency) and sleep apnea therapy efficacy.

In some examples, a target intensity level of stimulation energy is selected, determined, implemented, etc. without regard to intentionally establishing a discomfort threshold of the patient and/or an arousal threshold (such as in response to such stimulation). Stated differently, in at least some examples, a target intensity level of stimulation may be implemented to provide the desired efficacious therapeutic effect in reducing SDB without attempting to adjust or increase the target intensity level according to (or relative to) a discomfort threshold and/or an arousal threshold.

In some examples, the treatment period (during which stimulation may be applied at least part of the time) may comprise a period of time beginning with the patient turning on the therapy device and ending with the patient turning off the device. In some examples, the treatment period may comprise a selectable, predetermined start time (e.g., 10 p.m.) and selectable, predetermined stop time (e.g., 6 a.m.). In some examples, the treatment period may comprise a period of time between an auto-detected initiation of sleep and auto-detected awake-from-sleep time. With this in mind, the treatment period corresponds to a period during which a patient is sleeping such that the stimulation of the upper airway patency-related nerve and/or central sleep apnea-related nerve is generally not perceived by the patient and so that the stimulation coincides with the patient behavior (e.g., sleeping) during which the sleep disordered breathing behavior (e.g., central or obstructive sleep apnea) would be expected to occur.

Information related to the treatment period, in some examples, may be input to the data model and/or otherwise used by the care usage engine to set at least one data event parameter, such as the care usage engine 1106 illustrated by FIGS. 30A-30B.

Some non-limiting examples of such devices and methods to recognize and detect the various features and patterns associated with respiratory effort and flow limitations include, but are not limited to: U.S. Pat. No. 8,938,299, issued Jan. 20, 2015, and entitled “SYSTEM FOR TREATING SLEEP DISORDERED BREATHING”; U.S. Pat. No. 5,944,680, issued Aug. 31, 1999, and entitled “RESPIRATORY EFFORT DETECTION METHOD AND APPARATUS”; and U.S. Pat. No. 5,522,862, issued Jun. 4, 1996, and entitled “METHOD AND APPARATUS FOR TREATING OBSTRUCTIVE SLEEP APNEA”, the entire teachings of each are hereby incorporated by reference herein in their entireties.

Moreover, in some examples various stimulation methods may be applied to treat obstructive sleep apnea, which include but are not limited to: U.S. Pat. No. 10,583,297, issued Mar. 10, 2020, and entitled “METHOD AND SYSTEM FOR APPLYING STIMULATION IN TREATING SLEEP DISORDERED BREATHING”; U.S. Pat. No. 8,938,299, issued Jan. 20, 2015, and entitled “SYSTEM FOR TREATING SLEEP DISORDERED BREATHING”; U.S. Pat. No. 5,944,680, issued Aug. 31, 1999, and entitled “RESPIRATORY EFFORT DETECTION METHOD AND APPARATUS”; and U.S. Patent Publication No. 2018/0117316, published May 3, 2018, and entitled “STIMULATION FOR TREATING SLEEP DISORDERED BREATHING”, the entire teachings of each are hereby incorporated by reference herein in their entireties.

In some examples, the example stimulation electrode(s) 1412 shown in FIG. 31 may comprise at least some of substantially the same features and attributes as described in: U.S. Pat. No. 8,340,785, issued on Dec. 25, 2012, and entitled “SELF EXPANDING ELECTRODE CUFF”; U.S. Pat. No. 9,227,053, issued on Jan. 5, 2016, and entitled “SELF EXPANDING ELECTRODE CUFF”; U.S. Pat. No. 8,934,992, issued on Jan. 13, 2015, and entitled “NERVE CUFF”; and U.S. Patent Publication No. 2020/0230412, published on Jul. 23, 2020, and entitled “CUFF ELECTRODE”, the entire teachings of which are each incorporated herein by reference in their entireties. Moreover, in some examples a stimulation lead 1417, which may comprise one example implementation of a stimulation element, may comprise at least some of substantially the same features and attributes as the stimulation lead described in U.S. Pat. No. 6,572,543, issued Jun. 3, 2003, and entitled “SENSOR, METHOD OF SENSOR IMPLANT AND SYSTEM FOR TREATMENT OF RESPIRATORY DISORDERS”, the entire teachings of which is incorporated herein by reference in its entirety.

In some examples, the stimulation electrode 1412 may be delivered transvenously, percutaneously, etc. In some such examples, a transvenous approach may comprise at least some of substantially the same features and attributes as described in U.S. Pat. No. 9,889,299, issued Feb. 13, 2018, entitled “TRANSVENOUS METHOD OF TREATING SLEEP APNEA”, and which is hereby incorporated by reference in its entirety. In some such examples, a percutaneous approach may comprise at least some of substantially the same features and attributes as described in U.S. Pat. No. 9,486,628, issued Nov. 8, 2016, and entitled “PERCUTANEOUS ACCESS FOR SYSTEMS AND METHODS OF TREATING SLEEP APNEA”, the entire teachings of which is incorporated herein by reference in its entirety.

As further shown in the diagram of FIG. 31, in some examples, the device 1411 may be implemented with additional sensors 1420, 1430 to sense additional physiologic data, such as but not limited to, further respiratory information via sensing transthoracic bio-impedance, pressure sensing, etc. in order to complement the respiration information sensed via an acceleration sensor. In some examples, one or both of the sensors 1420, 1430 may comprise sensor electrodes. In some examples, stimulation electrode 1412 also may act, in some examples, as a sensing electrode. In some examples, at least a portion of housing of the IPG 1433 also may comprise a sensor or at least an electrically conductive portion (e.g., electrode) to work in cooperation with sensing electrodes to implement at least some sensing arrangements to sense bioimpedance, ECG, etc.

However, examples are not so limited and may be directed to other neurostimulation devices and cardiac care devices which may detect cardiac signals and provide atrial chamber stimulation therapy. For example, the IMD may include or be coupled to an implantable leads using to sense left and right atrial and ventricular cardiac signals. The electronics assembly of the IMD processes or monitors the cardiac signals and provides stimulation signals using a pulse generator and the implantable leads.

FIG. 32 is a diagram schematically representing an example IMD 1419A comprising at least some of substantially the same features and attributes as the device 1411 in FIG. 31, except with the IPG 1433 implemented as a microstimulator 1419B. In some examples, the microstimulator 1419B may be chronically implanted (e.g., percutaneously, subcutaneously, transvenously, etc.) in a head-and-neck region 1403 as shown in FIG. 32, or in a pectoral region 1401. In some examples, as part of the IMD 1419A, the microstimulator 1419B may be in wired or wireless communication with stimulation electrode 1412. In some examples, as part of the IMD 1419A, the microstimulator 1419B also may incorporate sensor 1435 or be in wireless or wired communication with a sensor 1435 located separately from a body of the microstimulator 1419B. When wireless communication is employed for sensing and/or stimulation, the microstimulator 1419B may be referred to as leadless implantable medical device for purposes of sensing and/or stimulation. In some examples, the microstimulator 1419B may be in close proximity to a target nerve 1405.

In some examples, the microstimulator 1419B (and associated elements) and/or IMD 1419A may comprise at least some of substantially the same features and attributes as described and illustrated within: U.S. Patent Publication No. 2020/0254249, published on Aug. 8, 2020, and entitled “MICROSTIMULATION SLEEP DISORDERED BREATHING (SDB) THERAPY DEVICE”; and U.S. Patent Publication No. 2020/0391028, published on Dec. 17, 2020, and entitled “IMPLANT-ACCESS INCISION AND SENSING FOR SLEEP DISORDERED BREATHING (SBD) CARE”, the entire teachings of which are incorporated herein by reference in their entireties.

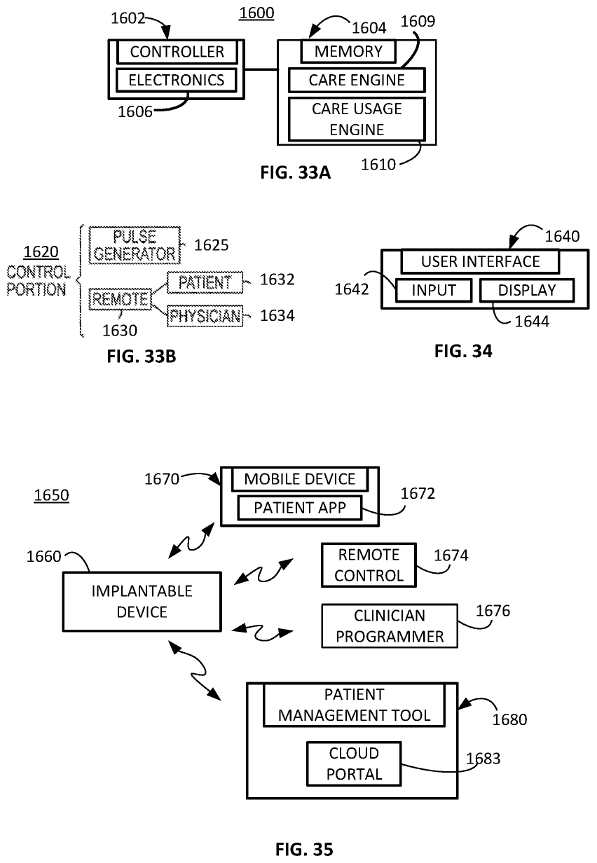

As implicated by the above description, one or both of the IMD and the external device includes a controller, control unit, or control portion that prompts performance of designated actions. FIG. 33A is a block diagram schematically representing an example control portion 1600. In some examples, the control portion 1600 includes a controller 1602 and a memory 1604. In some examples, the control portion 1600 provides one example implementation of a control portion forming a part of, implementing, and/or managing any one of devices, systems, assemblies, circuitry, managers, engines, functions, parameters, sensors, electrodes, modules, and/or methods, as represented throughout the present disclosure in association with FIGS. 1-32.

In general terms, the controller 1602 of the control portion 1600 comprises an electronics assembly 1606 (e.g., at least one processor, microprocessor, integrated circuits and logic, etc.) and associated memories or storage devices. The controller 1602 is electrically couplable to, and in communication with, the memory 1604 to generate control signals to direct operation of at least some the devices, systems, assemblies, circuitry, managers, modules, engines, functions, parameters, sensors, electrodes, and/or methods, as represented throughout the present disclosure. In some examples, these generated control signals include, but are not limited to, employing the care usage engine 1610 of an IMD which may be a software program stored on the memory 1604 (which may be stored on another storage device and loaded onto the memory 1604), and executed by the electronics assembly 1606 to set at least one data event parameter. In addition, and in some examples, these generated control signals include, but are not limited to, employing the care engine 1609 stored in the memory 1604 to at least manage care provided to the patient, for example cardiac therapy or therapy for sleep disordered breathing, in at least some examples of the present disclosure. It will be further understood that the control portion 1600 (or another control portion) may also be employed to operate general functions of the various care devices/systems described throughout the present disclosure. In some examples, the care usage engine 1610 and the care engine 1609 may include at least some of substantially the same features as described by the care usage engine 1106 and the care engine 1108 of at least FIGS. 30A-30B. In at least some examples, the care usage engine 1610 and the care engine 1609 may involve sensing and/or stimulating features directed to sensing and/or simulating for urinary and/or pelvic disorders using at least some of substantially the same features and attributes as described within PCT Patent Publication No. WO2020243104, published on Dec. 3, 2020, and entitled “SYSTEMS AND METHODS FOR TREATING INCONTINENCE”, the entire teachings of which is incorporated herein by reference in its entirety.

In response to or based upon commands received via a user interface (e.g., user interface 1640 in FIG. 34) and/or via machine readable instructions, controller 1602 generates control signals as described above in accordance with at least some of the examples of the present disclosure. In some examples, controller 1602 is embodied in a general purpose computing device while in some examples, controller 1602 is incorporated into or associated with at least some of the sensors, sensing element, respiration determination elements, stimulation elements, power/control elements (e.g., pulse generators), devices, user interfaces, instructions, information, engines, functions, actions, and/or method, etc. as described throughout examples of the present disclosure.

For purposes of this application, in reference to the controller 1602, the term “processor” shall mean a presently developed or future developed processor (or processing resources) that executes machine readable instructions contained in a memory. In some examples, execution of the machine readable instructions, such as those provided via memory 1604 of control portion 1600 cause the processor to perform the above-identified actions, such as operating controller 1602 to implement the sensing, monitoring, identifying the care cycle, stimulation, treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1604. In some examples, the machine readable instructions may comprise a sequence of instructions, a processor-executable machine learning model, or the like. In some examples, memory 1604 comprises a computer readable tangible medium providing non-volatile storage of the machine readable instructions executable by a process of controller 1602. In some examples, the computer readable tangible medium may sometimes be referred to as, and/or comprise at least a portion of, a computer program product. In some examples, hard wired circuitry may be used in place of or in combination with machine readable instructions to implement the functions described. For example, controller 1602 may be embodied as part of at least one application-specific integrated circuit (ASIC), at least one field-programmable gate array (FPGA), and/or the like. In at least some examples, the controller 1602 is not limited to any specific combination of hardware circuitry and machine readable instructions, nor limited to any particular source for the machine readable instructions executed by the controller 1602.

In some examples, control portion 1600 may be entirely implemented within or by a stand-alone device.

In some examples, the control portion 1600 may be partially implemented in one of the sensors, sensing element, respiration determination elements, monitoring devices, stimulation devices, IMDs (or portions thereof), etc. and partially implemented in a computing resource (e.g., at least one external resource) separate from, and independent of, the IMDs (or portions thereof) but in communication with the IMDs (or portions thereof). For instance, in some examples control portion 1600 may be implemented via a server accessible via the cloud and/or other network pathways. In some examples, the control portion 1600 may be distributed or apportioned among multiple devices or resources such as among a server, an apnea treatment device (or portion thereof), and/or a user interface.

In some examples, control portion 1600 includes, and/or is in communication with, a user interface 1640 as shown in FIG. 34.

FIG. 33B is a diagram schematically illustrating at least some example arrangements of a control portion 1620 by which the control portion 1600 (FIG. 33A) may be implemented. In some examples, control portion 1620 is entirely implemented within or by an IPG assembly 1625, which has at least some of substantially the same features and attributes as a pulse generator (e.g., power/control element) as previously described throughout the present disclosure. In some examples, control portion 1620 is entirely implemented within or by a remote control 1630 (e.g., a programmer) external to the patient's body, such as a patient control 1632 and/or a physician control 1634. In some examples, the control portion 1600 is partially implemented in the IPG assembly 1625 and partially implemented in the remote control 1630 (at least one of patient control 1632 and physician control 1634).

FIG. 34 is a block diagram schematically representing a user interface 1640. In some examples, user interface 1640 forms part of and/or is accessible via a device external to the patient and by which the IMD system may be at least partially controlled and/or monitored. The external device which hosts user interface 1640 may be a patient remote (e.g., 1632 in FIG. 33B), a physician remote (e.g., 1634 in FIG. 33B) and/or a clinician portal. In some examples, user interface 1640 comprises a user interface or other display that provides for the simultaneous display, activation, and/or operation of at least some of the sensors, sensing element, respiration determination elements, stimulation elements, power/control elements (e.g., pulse generators), devices, user interfaces, instructions, information, modules, engines, functions, actions, and/or method, etc., as described in association with FIGS. 1-33B. In some examples, at least some portions or aspects of the user interface 1640 are provided via a graphical user interface (GUI), and may comprise a display 1644 and input 1642.

FIG. 35 is a block diagram 1650 which schematically represents some example implementations by which an IMD 1660 (e.g., IMD 22 (e.g., an IPG), implantable sensing monitor, and the like) may communicate wirelessly with external circuitry outside the patient. As described above, the controller and/or control portion of the IMD 1660 illustrated in FIG. 38 may be implemented by components of the IMD 1660, components of external circuitry, such as external devices (e.g., mobile device 1670, patient remote control 1674, a clinician programmer 1676, and a patient management tool 1680), and various combinations thereof. As shown in FIG. 41, in some examples, the IMD 1660 may communicate with at least one of patient application 1672 on a mobile device 1670, a patient remote control 1674, a clinician programmer 1676, and a patient management tool 1680. The patient management tool 1680 may be implemented via a cloud-based portal 1683, the patient application 1672, and/or the patient remote control 1674. Among other types of data, these communication arrangements enable the IMD 1660 to communicate, display, manage, etc. the data events and data event parameters, as well as to allow for adjustment to the various elements, portions, etc. of the example devices and methods if and where desired.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1-145. (canceled)

146. A method comprising:
identifying a care usage pattern of an implantable medical device (IMD) for a patient associated with the IMD; and
setting a data event parameter for the IMD based on the care usage pattern.

147. The method of claim 146, wherein setting the data event parameter comprises at least one of:
setting a polling interval for data communication by the IMD; and
setting a time window for data processing by the IMD.

148. The method of claim 146, further comprising optimizing communication latency by the IMD based on the data event parameter, wherein setting the data event parameter comprises setting a polling interval.

149. The method of claim 148, wherein the care usage pattern comprises at least one of:
expected care cycles of the IMD;
observed care cycles of the IMD; and
a current care cycle of the IMD.

150. The method of claim 148, wherein the polling interval is set based on a time of day.

151. The method of claim 148, wherein the polling interval is set based on at least one patient state.

152. The method of claim 151, further comprising determining a transition between different patient states among a plurality of patient states, including the at least one patient state, wherein the different patient states include at least one of:
different body positions; and
different sleep-wake statuses.

153. The method of claim 148, further comprising identifying a plurality of time windows during which the polling interval is to be set to below a threshold interval value based on the care usage pattern.

154. The method of claim 153, further comprising setting the polling interval to below the threshold interval value for at least a subset of the plurality of time windows based on a target total polling time.

155. The method of claim 154, wherein setting the polling interval below the threshold interval value for the at least subset of the plurality of time windows comprises:
time limiting a total amount of time in a day that the IMD is set to the polling interval below the threshold interval value based on the target total polling time.

156. The method of claim 154, wherein setting the polling interval below the threshold interval value for the at least subset of the plurality of time windows based on the target total polling time comprises:
limiting a total daily polling time of the IMD based on the target total polling time and allocating the total daily polling time based on the care usage pattern, wherein the care usage pattern includes observed communication patterns of the IMD.

157. The method of claim 146, wherein setting the data event parameter comprises adjusting a polling interval to different values with respect to different times of the day based on the care usage pattern to optimize communication latency and power performance of the IMD.

158. The method of claim 146, wherein setting the data event parameter comprises setting at least one time window for performing data processing by the IMD, and configuring the IMD to include the care usage pattern comprises programming the IMD to set the at least one time window based on the care usage pattern.

159. The method of claim 158, wherein batched data is processed by the IMD during the at least one time window when a battery parameter of the IMD is within a threshold level based on the care usage pattern, and the data processing comprising time-insensitive data processing operations.

160. The method of claim 159, further comprising performing time-sensitive data processing operations during and outside the at least one time window, wherein the time-sensitive data processing operations are associated with providing care by the IMD.

161. The method of claim 158, further comprising:
identifying a battery parameter of the IMD is within a threshold level during the at least one time window based on the care usage pattern; and
setting the at least one time window for performing the data processing.

162. The method of claim 146, wherein the care usage pattern comprises at least one of a care cycle and a data communication cycle, the care cycle being associated with at least one of a care usage time and an amount of care provided to the patient by the IMD, and the data communication cycle being associated with data communicated between the IMD and external circuitry.

163. The method of claim 146, further comprising:
identifying the care usage pattern by applying a data model to input comprising physiological data and care usage data over a period of time, wherein the care usage data comprises at least one of a type, an amount, a period of time, a time of day, and a day of the week associated with providing care by the IMD; and
sensing at least a portion of the physiological data via at least one implantable sensor in communication with the IMD, the physiological data comprising at least body movement and posture of the patient, wherein the at least one implantable sensor includes an acceleration sensor.

164. The method of claim 146, wherein the care usage pattern comprises expected care cycles of the IMD for the patient which are identified based on first data and setting the data event parameter comprises setting at least one polling interval or at least one time window for performing data processing by the IMD, the method further comprising:
transitioning from the at least one polling interval or the at least one time window to at least one revised polling interval or revised time window based on observed care cycles of the IMD for the patient, the observed care cycles being identified based on second data.

165. The method of claim 164, wherein:
the first data comprises at least one of:
literature data;
input from a medical caregiver;
demographic data associated with a plurality of representative patients; and
input from the patient; and
the second data comprises physiological data and care usage data sensed over a period of time via at least one implantable sensor in communication with the IMD.
